(19) United States
(12) Patent Application Publication
(10) Pub. No.: US 2023/0200806 A1
(43) Pub. Date: Jun. 29, 2023

(54) TRACTION TOOL, TRACTION SYSTEM, AND TRACTION METHOD AND SUTURING METHOD FOR SUTURE THREAD

(71) Applicant: Olympus Corporation, Tokyo (JP)

(72) Inventors: Hiroyuki MORISHITA, Hachioji-shi (JP); Kunihide KAJI, Hachioji-shi (JP); Keita OZAWA, Hino-shi (JP); Tsutomu OKADA, Tachikawa-shi (JP); Masatoshi TONOMURA, Koganei-shi (JP); Nobuko MATSUO, Tokyo (JP); Kosuke OKA, Tokyo (JP)

(21) Appl. No.: 18/179,037

(22) Filed: Mar. 6, 2023

Related U.S. Application Data
(63) Continuation of application No. PCT/JP2020/038591, filed on Oct. 13, 2020.

(51) Int. Cl. A61B 17/04

(52) U.S. Cl. CPC A61B 17/0469; A61B 17/0483

(57) ABSTRACT

A traction tool for performing traction, of a suture thread under a flexible endoscope is provided. The traction toot includes a first holder configured to hold the suture thread or a suture needle attached to the suture thread; a second holder having an engaging surface configured to engage the suture, thread such that the suture thread is capable of advancing and retracting and configured to release an engagement with the suture thread; and a guide member holding the first holder and the second holder such that the first holder and the second holder are relatively movable.

### TECHNICAL FIELD

The present disclosure relates to a traction tool, a traction system, and a traction method and a suturing method for a suture thread. This application is a continuation application based on International Patent Application No. PCT/JP2020/038591 filed on Oct. 13, 2020, the contents of the PCT international application is incorporated herein by reference.

### BACKGROUND ART

In the related art, a procedure of suturing a wound inside a hollow organ such as the digestive tract under observation using an endoscope has been performed. In such a procedure, a tool such as an endoscopic needle holder allowing needle work to be performed by grasping a suture needle has been used.

United States Patent Application, Publication No. 2018/042602 discloses an endoscopic suturing tool. The suturing tool disclosed in United States Patent Application, Publication No. 2018/042602 pulls a tissue and pierces a suture needle to the pulled out tissue. The suturing tool hooks a suture thread to a tip of the suture needle which has penetrated the tissue and sutures the tissue by returning the, suture needle to an original position.

### SUMMARY OF INVENTION

### Technical Problem

In consideration of the foregoing circumstances, an object of the present disclosure is to provide a traction tool, a traction system, and a traction method and a suturing method for a suture thread capable of sufficiently stitching a wound by pulling a suture thread attached to a suture needle in suturing work inside a confined hollow organ.

### Solution to Problem

In order to resolve the foregoing problems, the present disclosure proposes the following means.

A traction tool according to a first aspect of the present disclosure performs traction of a suture thread under a flexible endoscope. The traction tool includes: a first holder configured to hold the suture thread or a suture needle attached to the suture thread; a second holder having an engaging surface configured to engage the suture thread such that the suture thread is capable of advancing and retracting and configured to release an engagement with the suture thread; and a guide member holding the first holder and the second holder such that the first holder and the second holder are relatively movable.

A traction tool according to a second aspect of the present disclosure performs traction of a suture thread under a flexible endoscope. The traction tool includes a sheath; a hard portion provided at a distal end of the sheath; a first holder having a first grasp member disposed in the hard portion and a second grasp member provided in the hard portion so as to be turnable, and the first holder being configured to grasp a needle, which is attached to the suture thread, between the first grasp member and the second grasp member; and a second holder inserted into the hard portion, and configured to hold the suture thread such that the suture thread is capable of advancing and retracting.

A traction method for a suture thread according to a third aspect of the present disclosure uses the tool described above. The traction method includes a step of pulling the suture thread by relatively moving the first holder and the second holder, the first holder holding the suture thread or a suture needle fixed to the suture thread, and the second holder holding the suture thread such that the suture thread is capable of advancing and retracting.

A suturing method according to a fourth aspect of the present disclosure closes a wound formed in a lumen wall of a hollow organ using a tool including a first holder configured to hold a suture thread or a suture needle attached to the suture thread, and a second holder configured to hold the suture thread such that the suture thread is capable of advancing and retracting. The suturing method includes a first step of threading a first edge portion of the wound with the suture thread, a second step of threading the suture thread to a second edge portion of the wound, the suture thread extending from the first edge portion, and the second edge portion being existed at a position facing the first edge portion with the wound interposed therebetween, a third step of fixing the suture needle or a first part of the suture thread extending from the second edge portion to the lumen wall and holding a second part, which extends between the suture needle or the first portion and the second edge portion, of the suture thread by the second holder such that the suture thread is capable of advancing and retracting, and a fourth step of pulling the suture thread by relatively moving the first holder and the second holder.

### BRIEF DESCRIPTION OF DRAWINGS

FIG. 1 is a general view of a traction system according to a first embodiment.

FIG. 2 is a general view illustrating a needle holder in the traction system.

FIG. 3 is a cross-sectional view of a distal end of a sheath in the needle holder.

FIG. 4 is a perspective view illustrating a hard, portion in the needle holder.

FIG. 5 is a perspective view illustrating a distal end portion of an elongated member in the needle holder.

FIG. 6 is a view illustrating a connection part between the sheath and an operation portion in the needle holder in a cross-sectional view.

FIG. 7 is a view illustrating the distal end of the sheath and the operation portion in the needle holder.

FIG. 8 is a view illustrating a suture thread.

FIG. 9 is a view for explaining a first step and a second step performed using the traction system.

FIG. 10 is a view for explaining the first step and the second step performed using the traction system.

FIG. 11 is a view for explaining a third step performed using the traction system.

FIG. 12 is a view for explaining a fourth step performed using the traction system.

FIG. 13 is a view illustrating a wound after the fourth step.

FIG. 14 is a view illustrating a needle holder having a modified example of the elongated member.

FIG. 15 is a view for explaining the third step performed using the needle holder.

FIG. 16 is a view for explaining the fourth step performed using the needle holder.

FIG. 17 is a view illustrating the wound after the fourth step performed using the needle holder.

FIG. 18 is a view illustrating a needle holder having another modified example of the elongated member.

FIG. 19 is a front view of the hard portion of the needle holder viewed in a longitudinal axis direction.

FIG. 20 is a view for explaining the third step performed using the needle holder.

FIG. 21 is a view for explaining the fourth step performed using the needle holder.

FIG. 22 is a perspective view illustrating a needle holder and a distal end portion of an outer sheath of a traction system according to a second embodiment.

FIG. 23 is a view illustrating the needle holder and the outer sheath.

FIG. 24 is a view illustrating a connection part between the sheath and an operation portion in the needle holder in a cross-sectional view.

FIG. 25 is across-sectional view along line D-D in FIG. 23.

FIG. 26 is a view for explaining the third step performed using the traction system.

FIG. 27 is a view for explaining the fourth step performed using the traction system.

FIG. 28 is a view illustrating a modified example of the traction system.

FIG. 29 is a view for explaining the fourth step performed using the traction system.

FIG. 30 is a view for explaining the fourth step performed using the traction system.

FIG. 31 is a view illustrating another modified example of a traction system according to the second embodiment.

FIG. 32 is a view for explaining the fourth step performed using the traction system.

FIG. 33 is a view illustrating another modified example of a traction system according to the second embodiment.

FIG. 34 is a view for explaining the fourth step performed using the traction system.

FIG. 35 is a view illustrating another modified example of a traction system according to the second embodiment.

FIG. 36 is a view illustrating another modified example of a traction system according to the second embodiment.

FIG. 37 is a perspective view illustrating a distal end portion of the traction system according to a third embodiment.

FIG. 38 is a view for explaining; the fourth step performed using the traction system.

FIG. 39 is a view for explaining the fourth step performed using the traction system.

FIG. 40 is a view for explaining the fourth step performed using the traction system.

FIG. 41 is a side view of a modified example of a cap in the traction system.

FIG. 42 is a side view of the cap illustrated in FIG. 41 rotated 90 degrees around an axis.

FIG. 43 is a view for explaining the third step using the cap.

FIG. 44 is a view for explaining the fourth step using the cap.

FIG. 45 is a perspective view illustrating a distal end portion of a traction system according to a fourth embodiment.

FIG. 46 is a view for explaining the fourth step performed using the traction system.

### DESCRIPTION OF EMBODIMENTS

### First Embodiment

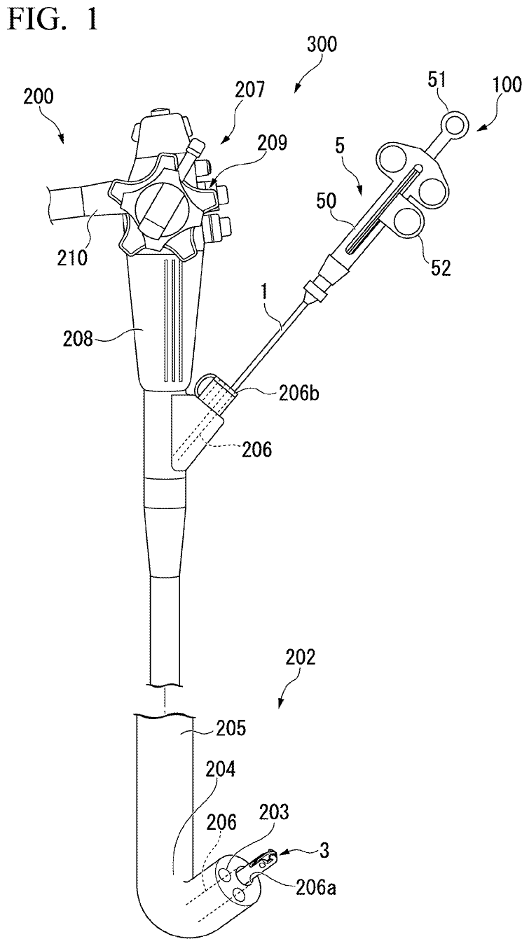

A traction system 300 according to a first embodiment of the present disclosure will be described with reference to FIGS. 1 to 13. FIG. 1 is a general view of the traction system 300 according to the present embodiment.

[Traction System 300]

As illustrated in FIG. 1, the traction system 300 includes a flexible endoscope 200 and a needle holder 100. The needle holder 100 is used so as to be inserted into the flexible endoscope 200.

[Flexible Endoscope 200]

As illustrated in FIG. 1, the flexible endoscope 200 includes an insertion body 202 inserted into a human body from a distal end thereof, and an operation portion 207 attached to a proximal end of the insertion body 202.

The insertion body 202 has an image capturing portion 203, a bent portion 204, and a flexible portion 205. From a distal end of the insertion body 202, the image capturing portion 203, the bent portion 204, and the flexible portion 205 are each arranged in this order. A channel 206 for inserting the needle holder 100 is provided inside the insertion body 202. A distal end opening portion 206a of the channel 206 is provided at the distal end of the insertion body 202.

For example, the image capturing portion 203 includes an image capturing element such as a CCD or CMOS, and configured to capture an image of a part of a treatment target. The image capturing portion 203 configured to capture an image of a grasper 3 of the needle holder 100 in a state in which the needle holder 100 protrudes from the distal end opening portion 206a of the channel 206.

The bent portion 204 is bent in accordance with an operation of the operation portion 207 by an operator. The flexible portion 205 is a tubular part having flexibility.

The operation portion 207 is connected to the flexible portion 205. The operation portion 207 has a grip 208, an input portion 209, a proximal end opening portion 206b of the channel 206, and a universal cord 210. The grip 208 is a part grasped by an operator. The input portion 209 receives an operational input for bending operation of the bent portion 204. The universal cord 210 outputs an image captured by the image capturing portion 203 to the outside. The universal cord 210 is connected to a display device such as a liquid crystal display via an image processing device including a processor and the like.

[Needle Holder 100]

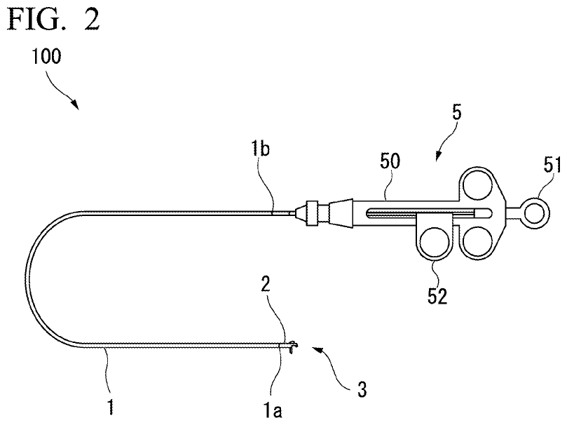

FIG. 2 is a general view illustrating the needle holder 100.

A needle holder (traction tool) 100 includes a sheath 1, a hard portion 2, a grasper (first holder) 3, an elongated member (second holder) 4, an operation portion 5, a first operation wire 6 for operating the grasper 3, and a second operation wire 7 for operating the elongated member 4.

The sheath 1 is an elongated member having flexibility and extending from a distal end 1a to a proximal end 1b. The sheath 1 has an outer diameter which is insertable into the channel 206 of the flexible endoscope 200. As illustrated in FIG. 1, in a state in which the sheath 1 is inserted into the channel 206, the distal end 1a of the sheath 1 can come in and out through the distal end opening portion 206a of the channel 206. The distal end 1a of the sheath 1 can enter the range of an image capturing visual field of the image capturing portion 203 of the flexible endoscope 200 and an image thereof is captured by the image capturing portion 203.

As illustrated in FIG. 2, the hard portion 2 is provided at the distal end 1a of the sheath 1. The grasper 3 is provided on the hard portion 2. The operation portion 5 is provided at a proximal end 1b of the sheath 1.

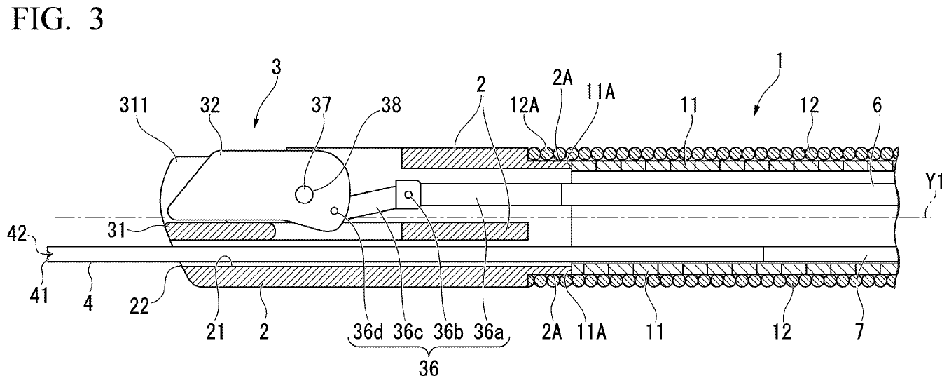

FIG. 3 is a cross-sectional view of the distal end 1a of the sheath 1.

The sheath 1 is a member endoscopically inserted into a body cavity. The sheath 1 has a first coil sheath 11 through which the first operation wire 6 and the second operation wire 7 are inserted, and a second coil sheath 12 through which the first coil sheath 11 is inserted.

The first coil, sheath 11 is a so-called single-strand coil sheath formed by tightly winding one metal based wire into a loop shape. The first coil sheath 11 has a compressive resistance with respect to the first operation wire 6 or the second operation wire 7 which has been inserted therethrough and suitably transmits an opening-closing operation of the grasper 3 or an advancing-retracting operation of the elongated member 4 via the operation portion 5 to the grasper 3. The first coil sheath 11 is not limited to a coil sheath and may be a resin tube such as a PEEK having an excellent compressive resistance.

The second coil sheath 12 is a so-called multi-strand coil sheath formed by arranging a plurality of metal-based wires in a radial direction and tightly winding them into a loop shape. The second coil sheath 12 suitably transmits an operation for rotating the hard portion 2 to the hard portion 2.

As illustrated in FIG. 3, the first coil sheath 11 is formed of a metal-based wire in which a transverse section of the metal-based wire has a rectangular shape. The second coil sheath 12 is formed of a metal-based wire in which a transverse section of the metal-based wire has a circular shape. The shapes of the transverse sections of the metal-based wires in the first coil sheath 11 and the second coil sheath 12 are not limited to these and may be suitably selected in accordance with a design value or the like of the sheath 1.

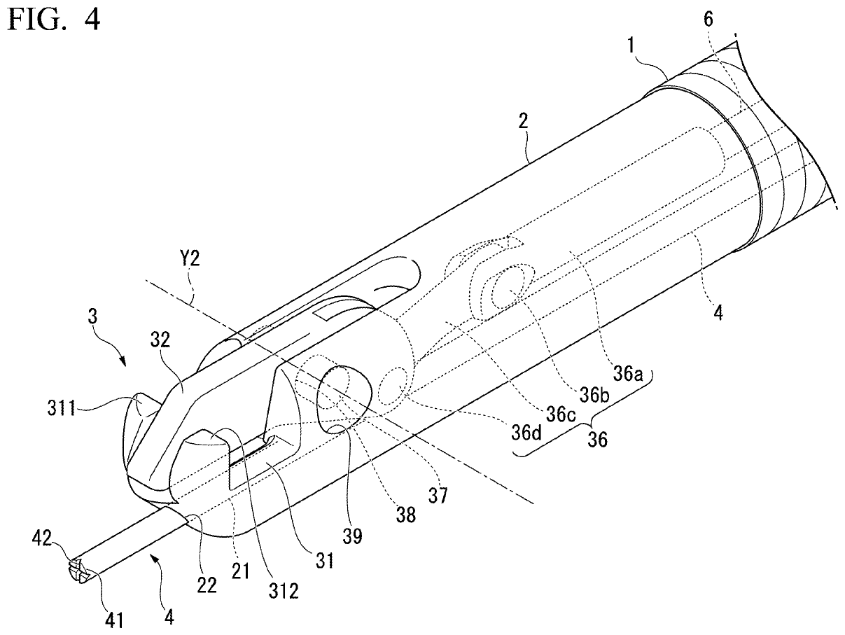

FIG. 4 is a perspective view illustrating the hard portion 2 of the needle holder 100.

The hard portion (guide member) 2 is formed to have substantially a cylindrical shape. The hard portion 2 is formed of a hard material such as a stainless steel material (SUS). The grasper 3 is provided in the distal end of the hard portion 2. The hard portion 2 is provided with a penetration hole 21 through which the elongated member 4 is inserted.

The penetration hole 21 is formed along a longitudinal axis Y1 of the sheath 1. A distal end opening 22 communicating with the penetration hole 21 is formed at the distal end of the hard portion 2. A distal end portion 41 of the elongated member 4 can come in and out through the distal end opening 22.

As illustrated in FIG. 3, a distal end 11A of the first coil sheath 11 is fixed to the proximal end of the hard portion 2 by laser welding, brazing, or the like.

The proximal end side of the hard portion 2 is formed to have a tubular shape and includes an outer surface 2A connected and fixed to the second coil sheath 12 on an outer surface thereof. The outer surface 2A on the proximal end side of the hard portion 2 is fixed to the second coil sheath 12 by laser welding, brazing, or the like.

A distal end 12A of the second coil sheath 12 fixed to the outer surface 2A of the hard portion 2 is unrotatable around an axis with respect to the hard portion 2 and is immovable in an axial direction with respect to the first coil sheath 11.

A connection form between the hard portion 2 and the sheath 1 is not limited to that described above. For example, a constitution in which the second coil sheath 12 is fixed to the outer surface and the first coil sheath 11 is fixed to an inner surface on the proximal end side of the hard portion 2 formed to have a tubular shape may be adopted. In addition, the shape of a part where the sheath 1 is fixed in the hard portion 2 may not have a tubular shape.

As illustrated in FIGS. 3 and 4, the grasper (first holder) 3 has a first grasp member 31, a second grasp member 32, and a link mechanism 36. The first grasp member 31 and the second grasp member 32 are constituted to be able to perform an opening/closing operation. The grasper 3 illustrated in FIG. 4 is in a closed state in which the first grasp member 31 and the second grasp member 32 are closed.

The first grasp member 31 is a part of the distal end portion of the hard portion 2. The first grasp member 31 extends along the longitudinal axis Y1 of the sheath 1. In the present embodiment, the first grasp member 31 and the hard portion 2 are integrally molded.

The second grasp member 32 is coupled to the hard portion 2 such that an opening/closing operation can be performed with respect to the first grasp member 31. A coupling shaft 37 is inserted into a penetration hole 38 formed in the second grasp member 32 and a penetration hole 39 formed in the hard portion 2, and therefore the second grasp member 32 is turnable coupled to the hard portion 2. The second grasp member 32 can turn around a longitudinal axis Y2 of the coupling shaft 37.

The first grasp member 31 has a first protrusion portion 311 and a second protrusion portion 312. The first protrusion portion 311 and the second protrusion portion 312 are provided in the distal end portion of the first grasp member 31 and protrude in a direction intersecting the longitudinal axis (center axis) Y1. The first protrusion portion 311 and the second protrusion portion 312 are provided as a pair with the longitudinal axis Y1 of the sheath 1 interposed therebetween, and the distal end portion of the second grasp member 32 is positioned between the first protrusion portion 311 and the second protrusion portion 312 in a state in which the first grasp member 31 and the second grasp member 32 are closed.

The link mechanism 36 is constituted of a first link member 36a, a first joint member 36b, a second link member 36c, and a second joint member 36d. The first link member 36a is coupled to the second link member 36c by the first joint member 36b. The second link member 36c is coupled to the second grasp member 32 by the second joint member 36d.

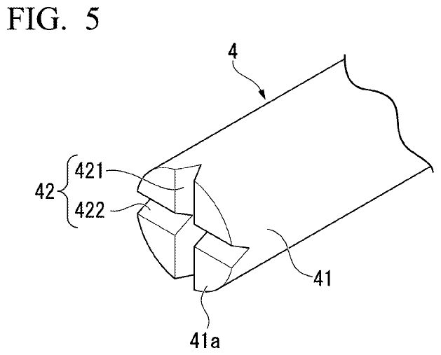

FIG. 5 is a perspective view illustrating the distal end portion 41 of the elongated member 4.

The elongated member (second holder) 4 is formed to have a round rod shape. The elongated member 4 is formed of a hard material such as a stainless steel material (SUS).

The distal end portion 41 of the elongated member 4 has a groove (hook portion, engaging surface) 42. For example, the groove 42 is constituted of a first groove 421 and a second groove 422. The first groove 421 and the second groove 422 are capable of engaging a suture thread S such that the suture thread S is capable of advancing and retracting.

The first groove 421 extends in the radial direction on a circular end surface 41a. The first groove 421 extends to an outer circumference of the distal end portion 41.

The second groove 422 extends in the radial direction on the circular end surface 41a. The second groove 422 extends to the outer circumference of the distal end portion 41.

The first groove 421 and the second groove 422 intersect each other on the end surface 41a of the distal end portion 41. In the present embodiment, the first groove 421 and the second groove 422 are orthogonal to each other.

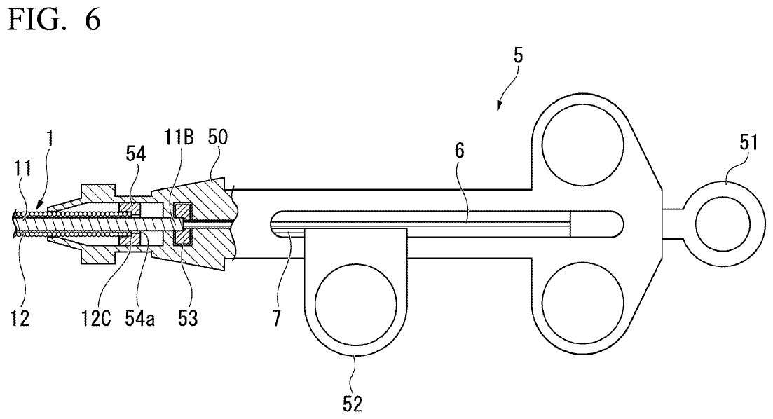

FIG. 6 is a view illustrating a connection part between the sheath 1 and the operation portion 5 in a cross-sectional view.

The operation portion (handle) 5 has an operation portion main body 50, a first slider 51, a second slider 52, an engage member (disk) 53 and a sliding member (key) 54.

A proximal end 12C of the second coil sheath 12 is fixed to the sliding member 54 inside the operation portion main body 50. A proximal end 11B of the first coil sheath 11 extending from the second coil sheath 12 is fixed to the engage member 53.

The first slider 51 is coupled such that the first slider 51 is advanceable and retractable with respect to the operation portion main body 50 and is capable of advancing and retracting in the axial direction of the operation portion main body 50. The first operation wire 6 extending from the first coil sheath 11 is connected to the first slider 51 through an inside of the operation portion main body 50.

The second slider 52 is coupled so as to be advanceable and retractable with respect to the operation portion main body 50. The second slider 52 is advanceable and retractable in the axial direction of the operation portion main body 50. The second operation wire 7 extending from the first coil sheath 11 is connected to the second slider 52 through the inside of the operation portion main body 50.

The engage member (disk) 53 is formed to have substantially a cylindrical shape. The proximal end 11B, which is fixed to both the engage member 53 and the engage member 53, of the first coil sheath 11 is attached such that the proximal end 11B is rotatable around the axis with respect to the operation portion main body 50 and is immovable relative to the operation portion main body 50 in the axial direction.

The sliding member (key) 54 is a member sliding inside the operation portion main body 50 in a longitudinal direction and has a penetration hole 54a through which the first coil sheath 11 is inserted. The sliding member 54 and the proximal end 12C of the second coil sheath 12 fixed to the sliding member 54 are attached so as to be unrotatable about the axis with respect to the operation portion main body 50 and so as to be movable in the axial direction with respect to the operation portion main body 50 and the first coil sheath 11.

If the operation portion main body 50 is rotated about the axis, the sliding member 54 and the second coil sheath 12 also rotate about the axis together with the operation portion main body 50.

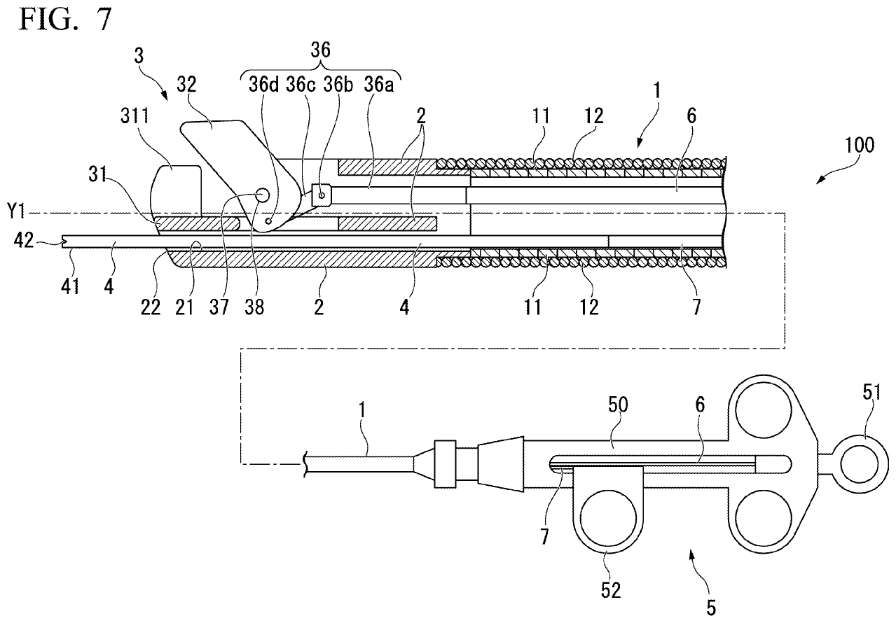

FIG. 7 is a view illustrating the distal end 1a of the sheath 1 and the operation portion 5.

The first operation wire 6 is disposed along the longitudinal axis Y1 of the sheath 1 inside the sheath 1. The first operation wire 6 is a flexible wire, and is capable of transmitting the amount of operation force from the operation portion 5.

A distal end of the first operation wire 6 is fixed to the first link member 36a of the link mechanism 36. The proximal end of the first operation wire 6 is connected to the first slider 51 of the operation portion 5. That is, the distal end of the first operation wire 6 and the second grasp member 32 are connected to each other via the link mechanism 36. The amount of operation force for operating an opening/closing operation of the second grasp member 32 with respect to the first grasp member 31 is transmitted from the operation portion 5 to the second grasp member 32 via the first operation wire 6 and the link mechanism 36.

Due to advance and retract of the first slider 51 with respect to the operation portion main body 50, the first operation wire 6 is capable of being made to advance and retract along the longitudinal axis Y1 of the sheath 1. The first operation wire 6 is capable of being pulled toward the operation portion 5 side by moving the first slider 51 toward the proximal side with respect to the operation portion main body 50.

When the first operation wire 6 is pulled toward the operation portion 5 side, the second grasp member 32 moves in a direction in to be closed with respect to the first grasp member 31. On the other hand, as illustrated in FIG. 7, when the first operation wire 6 is pushed out toward the grasper 3 side, the second grasp member 32 moves in a direction to be opened with respect to the first grasp member 31.

The second operation wire 7 is disposed along the longitudinal axis Y1 of the sheath 1 inside the sheath 1. The second operation wire 7 is a flexible wire, and is capable of transmitting the amount of operation force from the operation portion 5.

A distal end of the second operation wire 7 is fixed to a proximal end of the elongated member 4. The proximal end of the second operation wire 7 is connected to the second slider 52 of the operation portion 5. The amount of operation force for advancing and retracting with respect to the elongated member 4 is transmitted from the operation portion 5 to the elongated member 4 via the second operation wire 7.

The second operation wire 7 is capable of advancing and retracting along the longitudinal axis Y1 of the sheath 1 by advancing and retracting the second slider 52 along the operation portion main body 50. The second operation wire 7 is capable of being pulled toward the operation portion 5 side by moving the second slider 52 toward the proximal side along the operation portion main body 50.

As illustrated in FIG. 7, when the second operation wire 7 is pushed out toward the grasper 3 side, the distal end portion 41 of the elongated member 4 protrudes from through the distal end opening 22 of the hard portion 2. On the other hand, when the second operation wire 7 is pulled toward the operation portion 5 side, the distal end portion 41 of the elongated member 4 is accommodated in the penetration hole 21 of the hard portion 2.

[Method for Using Traction System 300]

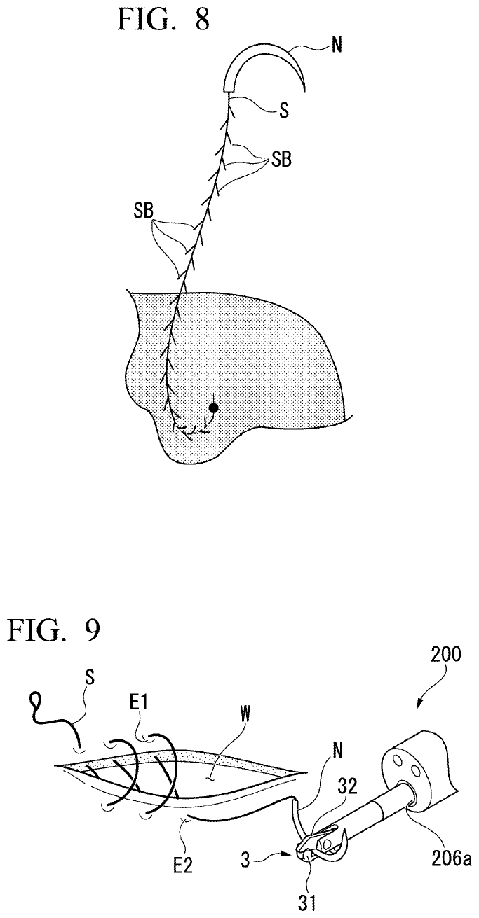

Next, with reference to FIGS. 8 to 12, a procedure of using the traction system 300 of the present embodiment (method for using the traction system 300) will be described. Specifically, a procedure of suturing a wound W formed in a lumen wall T inside a hollow organ such as the digestive tract using the needle holder 100 will be described. FIGS. 8 to 12 are views illustrating a method for using the needle holder 100.

FIG. 8 is a view illustrating the suture thread S.

The suture thread S used in the procedure described below has a plurality of barbs SB disposed side by side along the longitudinal axis. The suture thread S is allowed to move in only a direction in which a suture needle N is attached in a state in which the suture thread S is inserted into a biological tissue. Since the plurality of barbs SB are engaged with the biological tissue, the suture thread S is immovable in the opposite direction. In the diagrams except for FIG. 8, illustration of the barbs SB of the suture thread S is omitted.

An operator causes the grasper 3 of the needle holder 100 to protrude from the distal end opening portion 206a of the channel 206 of the flexible endoscope 200 before the flexible endoscope 200 is inserted into a hollow organ. The operator grasps the suture needle N with the grasper 3. The operator may grasp the suture needle N attached to the suture thread S with the grasper 3.

The operator inserts the flexible endoscope 200 into a hollow organ through a natural opening of a patient. The suture needle N or the suture thread S is introduced into a hollow organ in a state of being grasped by the grasper 3 which has protruded through the distal end opening portion 206a.

When the suture needle N grasped by the grasper 3 in an undesired state in direction or a position, the operator temporarily places the suture needle N on the lumen wall T and regrasps the suture needle N with the grasper 3. If the suture thread S is grasped when the suture needle N is introduced into the digestive tract, the suture needle N is also temporarily placed on the lumen wall T, and the suture needle N is regrasped by the grasper 3. The operator grasps the suture needle N with the grasper 3 by moving the first slider 51 toward the proximal side along the-operation portion main body 50.

[First Step]

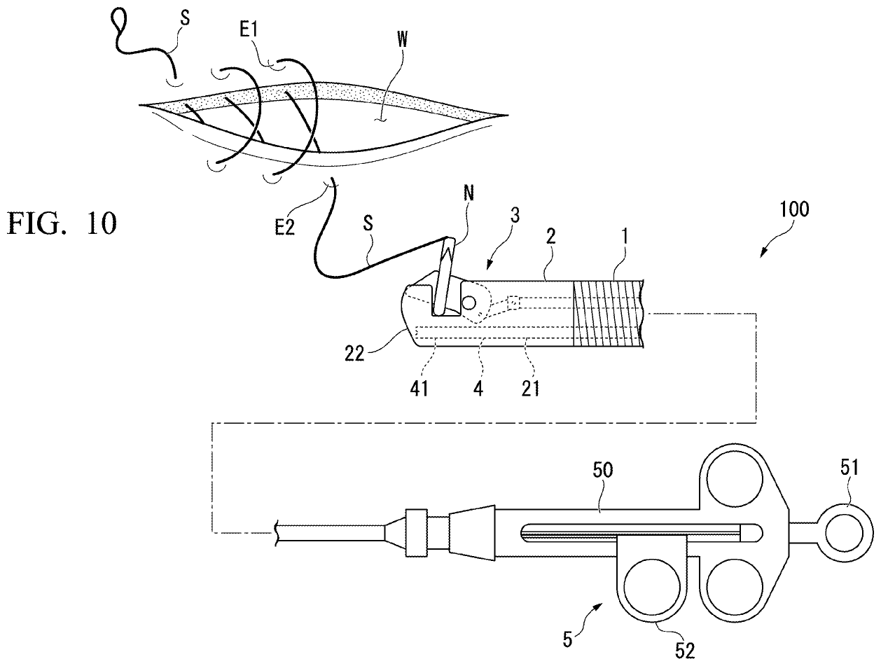

FIGS. 9 and 10 are explanatory views of a first step and a second step.

In the first step, while grasping the suture needle N with the grasper 3 caused to protrude through the distal end opening portion 206a, the operator punctures the suture needle N into the first edge portion E1 of the wound W formed in the lumen wall T inside a hollow organ to thread a first edge portion E1 with the suture thread S.

[Second Step]

In the second step, while grasping the suture needle N with the grasper 3, the operator threads a second edge portion E2 of the wound W formed in the lumen wall T inside the digestive tract with the suture thread S extending from the first edge portion E1 by puncturing the suture needle N into the second edge portion E2. The second edge portion E2 is an edge portion of the wound W and is at a position facing the first edge portion E1 with the wound W interposed therebetween.

The operator repeatedly performs the first step and the second step a plurality of times in accordance with the size of the wound W. The wound W illustrated in FIG. 9 indicates a state in which the first step and the second step have been performed three times.

As illustrated in FIG. 10, in the first step and the second step, it is preferable that the operator moves the second slider 52 toward the proximal side along the operation portion main body 50 and accommodates the distal end portion 41 of the elongated member 4 in the penetration hole 21 of the hard portion 2.

[Third Step]

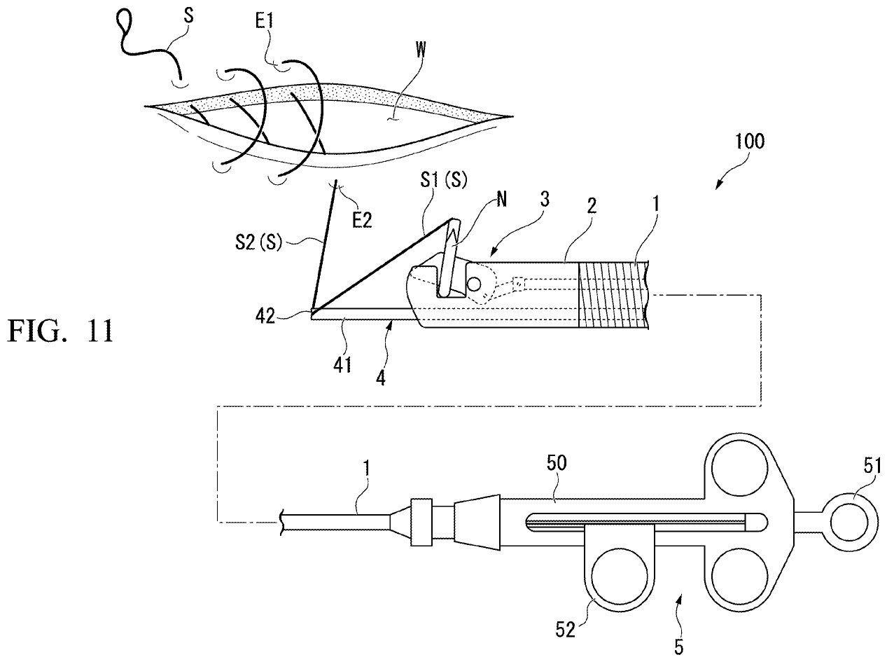

FIG. 11 is a view or explaining a third step.

The operator causes the distal end portion 41 of the elongated member 4 to protrude from a distal end of the penetration hole 21 of the hard portion 2 by moving the second slider 52 toward the distal end side with respect to the operation portion main body 50. In the third step, while holding a first part S1 of the suture thread S extending from the second edge portion E2 or the suture needle N with the grasper (first holder) 3, the operator engages (holds) a second part 52, which extends between the suture needle N or the first part S1 and the second edge portion E2, of the suture thread S with the distal end portion 41 of the elongated member (second holder) 4 so as, to be capable of advancing and retracting. Specifically, the operator engages the suture thread S which has entered the, inside of the groove 42 of the elongated membe (second holder) 4 so as to be capable of advancing and retracting.

[Fourth Step]

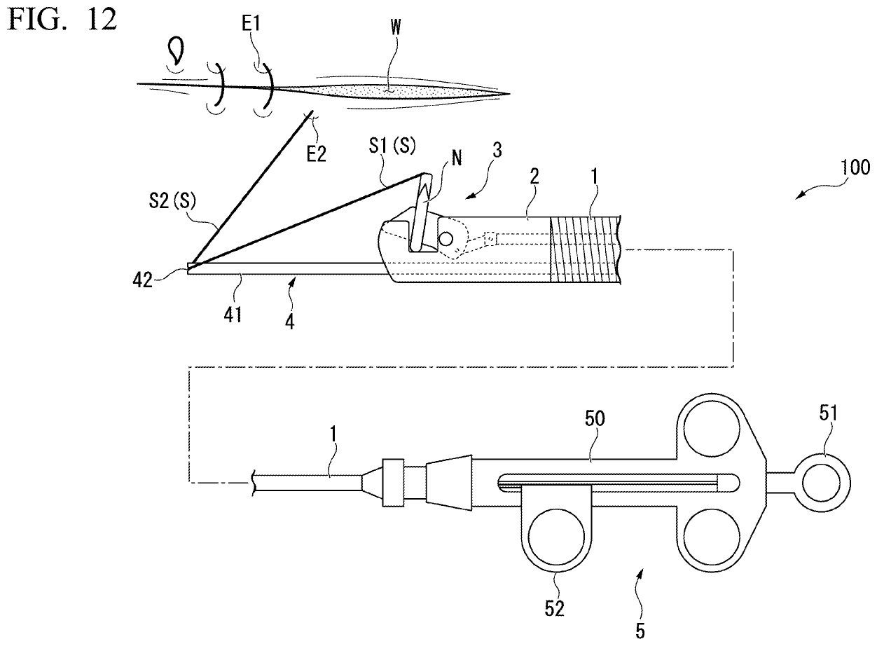

FIG. 12 is a view for explaining a fourth step.

In the fourth step, the operator pulls the suture thread S by relatively moving the grasper (first holder) 3 and the elongated, member (second holder) 4. Specifically, the operator advances the elongated member 4 with respect to the grasper 3 until the distal end portion 41 (groove 42) of the elongated member 4 is positioned closer to the distal side than the grasper 3 by moving the second slider 52 toward the distal end side with respect to the operation portion main body 50. In accordance with this operation, the distal end portion 41 (groove 42) of the elongated member (second holder) 4 in which the suture thread S has been engaged moves in a direction in which the distal end portion 41 becomes more distant from the grasper (first holder) 3 grasping the suture needle N and the second edge portion E2. As a result, a tension is applied to the suture thread S, and therefore the first edge portion E1 and the second edge portion E2 are tightened.

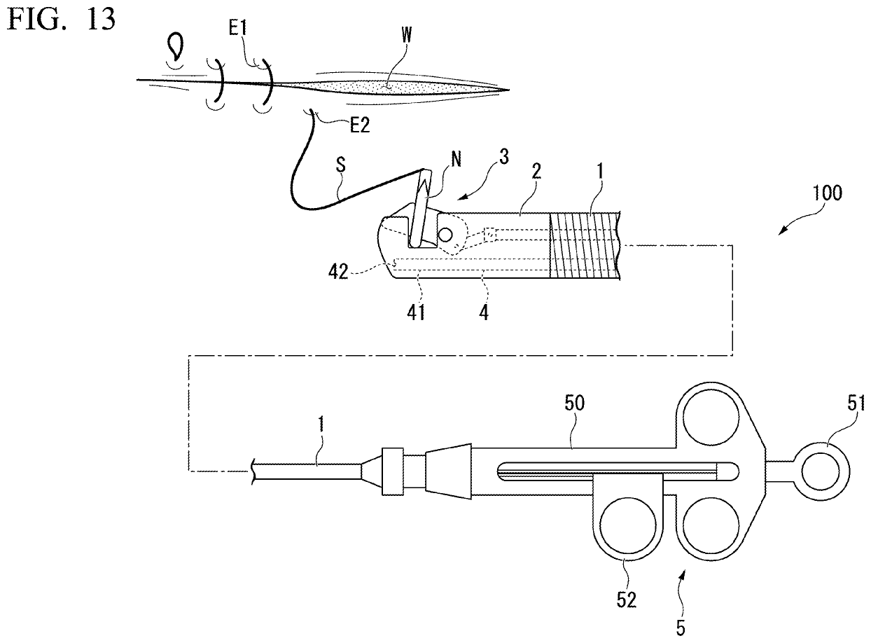

FIG. 13 is a view illustrating the wound W after the fourth step.

The operator retracts the elongated member 4 with respect to the grasper 3 by moving the second slider 52 toward the proximal side with respect to the operation portion main body 50. As a result, the tension applied to the suture thread S is canceled. Since the suture thread S has a plurality of barbs, the tightened state of the first edge portion E1 and the second edge portion E2 is maintained.

The operator, sutures the wound W which has not sutured by repeatedly performing the first step to the fourth step a plurality of times in accordance with the size of the wound W. The operator cuts the suture thread S, takes the cut suture thread S and the suture needle N out of the body, and ends the procedure.

According to the endoscopic needle holder 100 according to the present embodiment, for example, in suturing work inside a confined hollow organ such as a part near the pyloric part of the stomach, a wound is sufficiently stitched by pulling the suture thread S attached to the suture needle N.

According to the procedure using the endoscopic needle holder 100 (method of use) according to the present embodiment, when the suture thread S attached to the suture needle N is pulled, there is no need to move the grasper 3 grasping, the suture needle N. For this reason, it is easy for the operator to restart suturing work of the wound W after traction of the suture thread S.

Hereinabove, the first embodiment of the present disclosure has been described with reference to the drawings, but specific constitutions are not limited to this embodiment, and design change and the like within a range not departing from the gist of the present disclosure are also included therein. In addition, the constituent elements illustrated in the embodiment and the modified examples described above can be constituted to be suitably combined.

### MODIFIED EXAMPLE 1

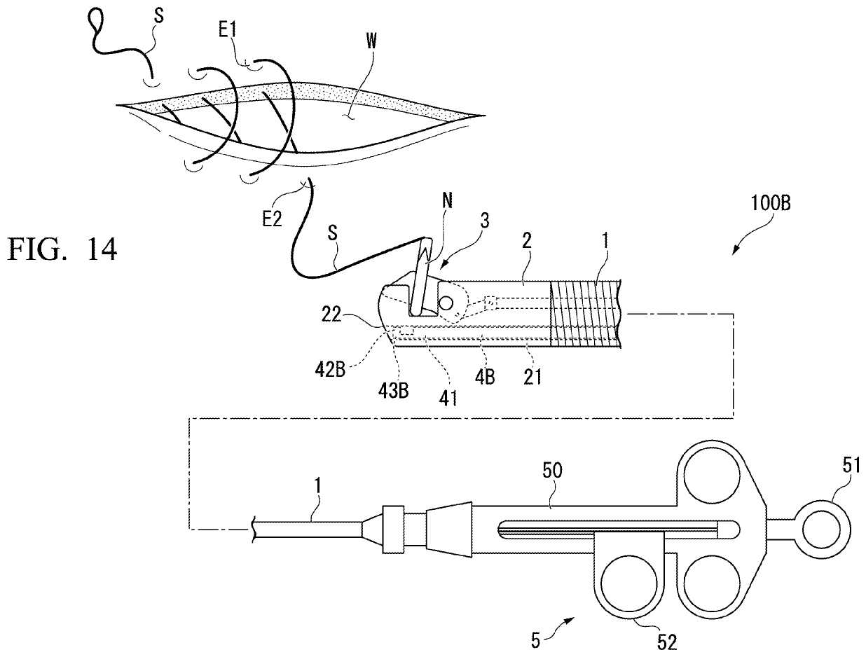

FIG. 14 is a view illustrating a needle holder 100B having an elongated member 4B that is a modified example of the elongated member 4. The distal end portion 41 of the elongated member 4B has a hook 43B. The hook 43B has a protrusion (hook portion, engaging surface) 42B extending in a direction intersecting the longitudinal axis of the elongated member 4B. The hook 43B engages (holds) the suture thread S so as to be advanceable and retractable. The hook 43B is configured to release an engagement with the suture thread. Specifically, the suture thread S is engaged by the protrusion 42B so as to be advanceable and retractable. For example, the hook 43B has an L-shape. As illustrated in FIG, 14, the operator performs the first step and the second step similar to those in the foregoing embodiment.

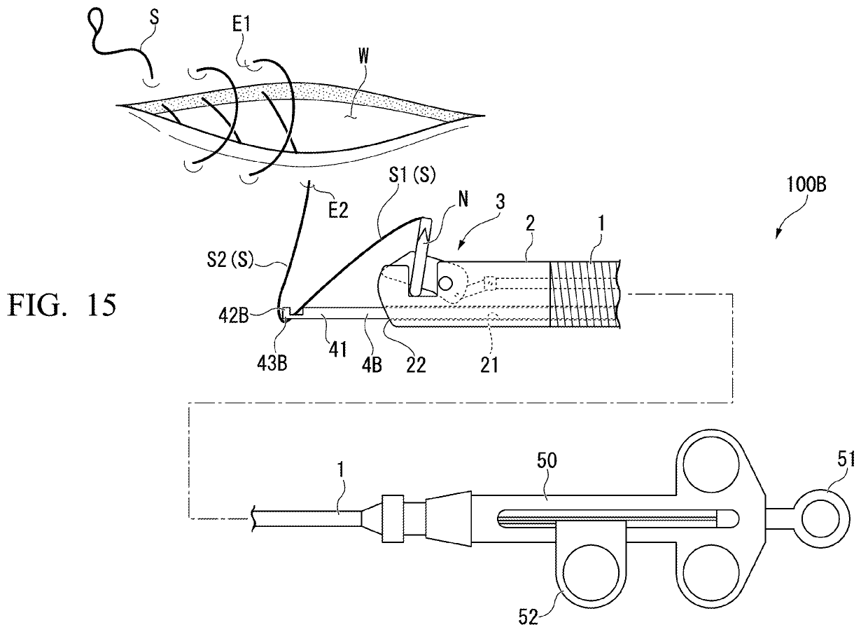

FIG. 15 is a view for explaining the third step performed using the needle holder 100B.

In the third step, the operator causes the distal end portion 41 of the elongated member 4B to protrude from the distal end of the penetration hole 21 of the hard portion 2 by slightly moving the second slider 52 toward the distal end side with respect to the operation portion main body 50. The operator engages (holds) the suture thread S with the hook 43B of the distal end portion 41.

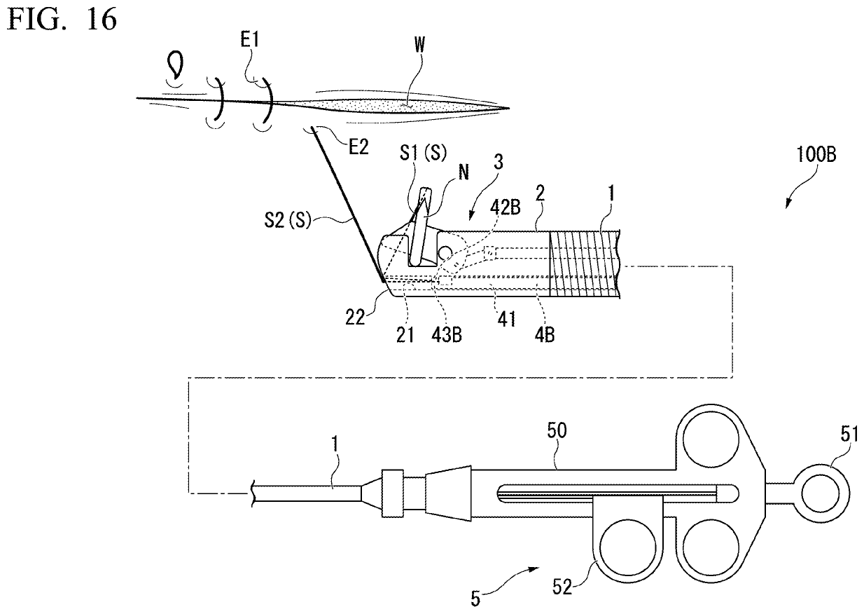

FIG. 16 is a view for explaining the fourth step performed using the needle holder 100B.

The operator retracts the elongated member 4B with respect toward the grasper 3 by moving the second slider 52 toward the proximal side with respect to the operation portion main body 50. In accordance with this operation, the hook 43B of the elongated member (second holder) 4B in which the suture thread S has been engaged moves in a direction in which the hook 43B becomes more distant from the grasper (first holder) 3 grasping the suture needle N and the second edge portion E2. As a result, a tension is applied to the suture thread S, and therefore the first edge portion E1 and the second edge portion E2 are tightened. When the elongated member 4B is retracted with respect to the grasper 3, the hook 43B is accommodated in the penetration hole 21 of the hard portion 2.

FIG. 17 is a view illustrating the wound W after the fourth step performed using the needle holder 100B.

The operator causes the distal end portion of the elongated member 4B to protrude from the distal end of the penetration hole 21 of the hard portion 2 by moving the second slider 52 to the distal end side again with respect to the operation portion main body 50. In this state, the operator can remove the suture thread S from the hook 43B. Next, as illustrated in FIG. 16, the operator accommodates the distal end portion 41 of the elongated member 4B in the penetration hole 21 of the hard portion 2 again by moving the second slider 52 toward the proximal side with respect to the operation portion main body 50.

According to the elongated member 4B that is a modified example of the elongated member 4, since the elongated member 4B has the hook 43B, the operator can reliably engage the suture thread S. The operator can move a part in which the suture thread S has been engaged in a direction in which it becomes more distant from the grasper 3 grasping the suture needle N and the second edge portion E2 even by retracting the elongated member 4B.

### MODIFIED EXAMPLE 2

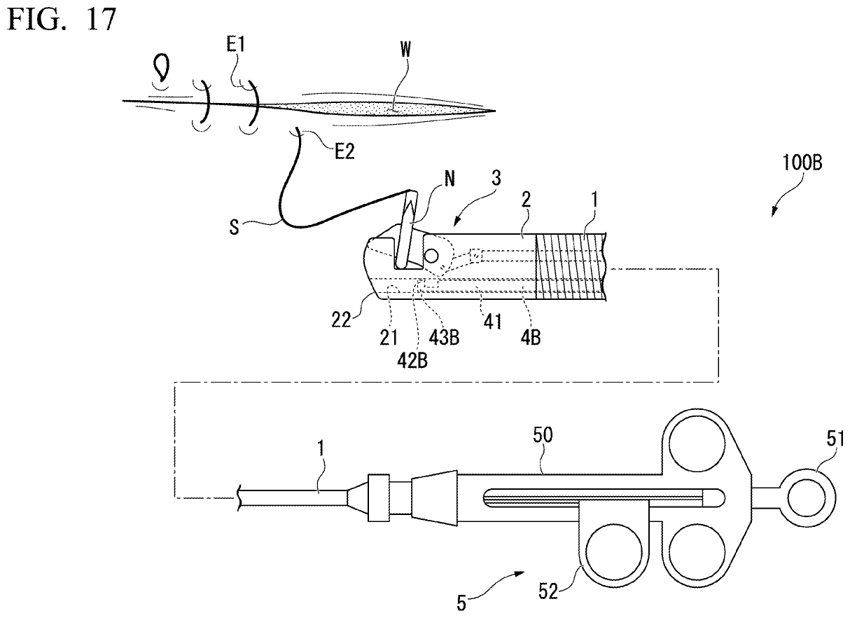

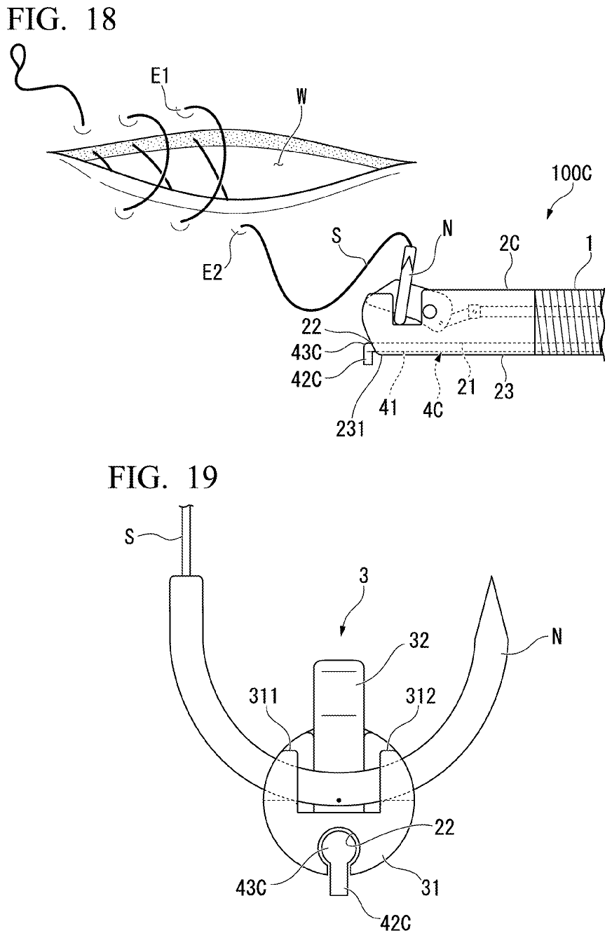

FIG. 18 is a view illustrating a needle holder 100C having an elongated member 4C that is a modified example of the elongated member 4. The distal end portion 41 of the elongated member 4C has a hook 43C. The hook 43C has a protrusion (hook portion, engaging surface) 42C extending in a direction intersecting the longitudinal axis of the elongated member 4C. The hook 43C engages (holds) the suture thread S so as to be advanceable and retractable. The hook 43C is configured to release an engagement with the suture thread. Specifically, the suture thread S is engaged with the protrusion 42C so as to be advanceable and retractable. For example, the hook 43C has an L-shape. The suture thread S is capable of being hooked to the hook 43C.

FIG. 19 is a front view of a hard portion 2C viewed in the longitudinal axis Y1 direction.

The needle holder 100C has the hard portion 2C that is a modified example of the hard portion 2. In the hard portion 2C, a slit 23 communicating with the penetration hole 21 extends along the longitudinal axis Y1 of the sheath 1. The protrusion 42C of the hook 43C can be inserted through the slit 23. A distal end 231 of the slit 23 is positioned in the hard portion 2C. The protrusion 42C of the hook 43C protrudes from the slit 23 to the outward side of the grasper (first holder) 3 and the hard portion 2C in the radial direction. As illustrated in FIG. 18, the operator performs the first step and the second step similar to those in the foregoing embodiment.

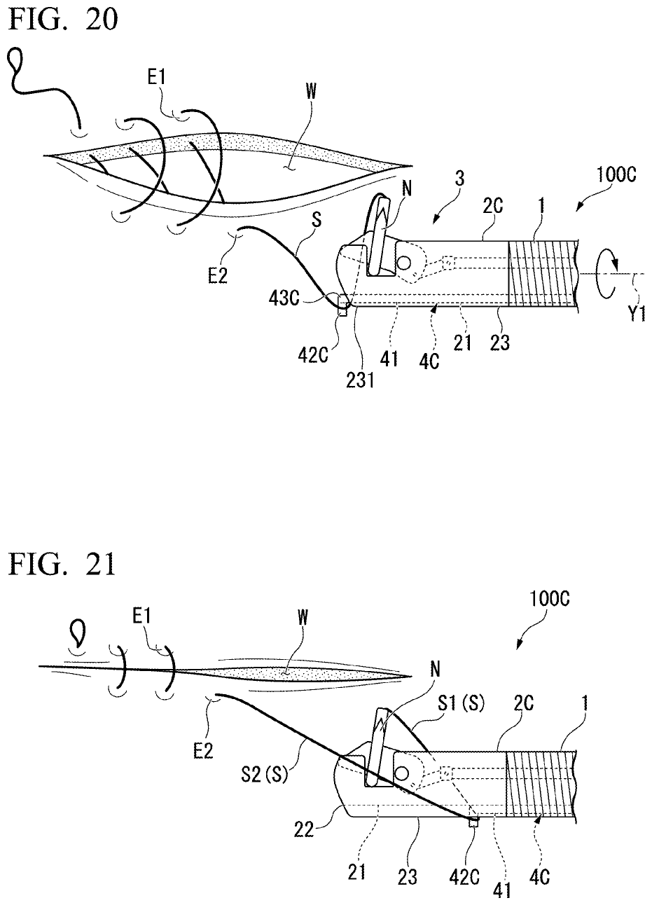

FIG. 20 is a view for explaining the third step performed using the needle holder 100C.

In the third step, the operator forms a loop with the suture thread S by rotating the needle holder 100C about the longitudinal axis Y1 of the sheath 1. Next, the operator engages (holds) the suture thread S by hooking the hook 43C of the elongated member 4C to the loop through retracting operation of the elongated member 4C.

FIG. 21 is a view for explaining the fourth step performed using the needle holder 100C.

The operator retracts the elongated member 4C with respect to the grasper 3 by moving the second slider 52 toward the proximal side with respect to the operation portion main body 50. In accordance with this operation, the hook 43C of the elongated member (second holder) 4C in which the suture thread S has been engaged moves in a direction in which it becomes more distant from the grasper (first holder) 3 grasping the suture needle N and the second edge portion E2. As a result, a tension is applied to the suture thread S, and therefore the first edge portion E1 and the second edge portion E2 are tightened.

According to the elongated member 4C that is a modified example of the elongated member 4, it has the hook 43C so that the operator can reliably engage the suture thread S. The operator can easily hook the hook 43C of the elongated member 4C to a loop formed by rotating the needle holder 100C about the longitudinal axis Y1 of the sheath 1.

### Second Embodiment

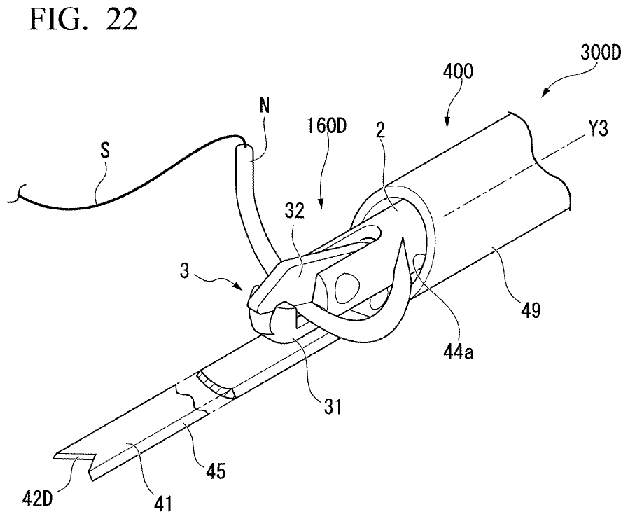

A traction system 300D according to a second embodiment of the present disclosure will be described with reference to FIGS. 22 to 27. In the following description, the same reference signs are applied to constituents common to those which have already been described, and duplicate description will be omitted.

[Traction System 300D]

FIG. 22 is a perspective view illustrating a needle holder 100D and a distal end portion of an outer sheath 400.

The traction system 300D includes the flexible endoscope 200, the needle holder 100D, and the outer sheath 400. The needle holder 100D is used so as to be inserted into the flexible endoscope 200 in a state of being inserted through the outer sheath 400.

[Outer Sheath 400]

The outer sheath (second holder, second holder, or tube) 400 is a cylinder member (guide member) 44 having flexibility. The outer sheath 400 has an extension portion (second holder) 45 at the distal end of the cylinder member 44. The cylinder member 44 has an inner diameter through which the needle holder 100D can be inserted. The needle holder 100D can protrude through a distal end opening 44a of the cylinder member 44. The extension portion 45 is formed to have a width smaller than the outer diameter of the cylinder member 11 and extends from the edge of the distal end opening of the cylinder member 44 along a longitudinal axis Y3 of the outer sheath 400. The shape of a transverse section of the extension portion 45 may have an arc shape or may have a circular shape or a plate shape. The distal end portion 41 of the extension portion 45 has a hook portion (engaging surface) 42D in which the suture thread S is capable of being engaged so as to be advanceable and retractable. The hook portion 42D is configured to release an engagement with the suture thread. The hook portion 42D has a recessed shape, and it may have a U-shape or a V-shape, for example.

[Needle Holder 100D]

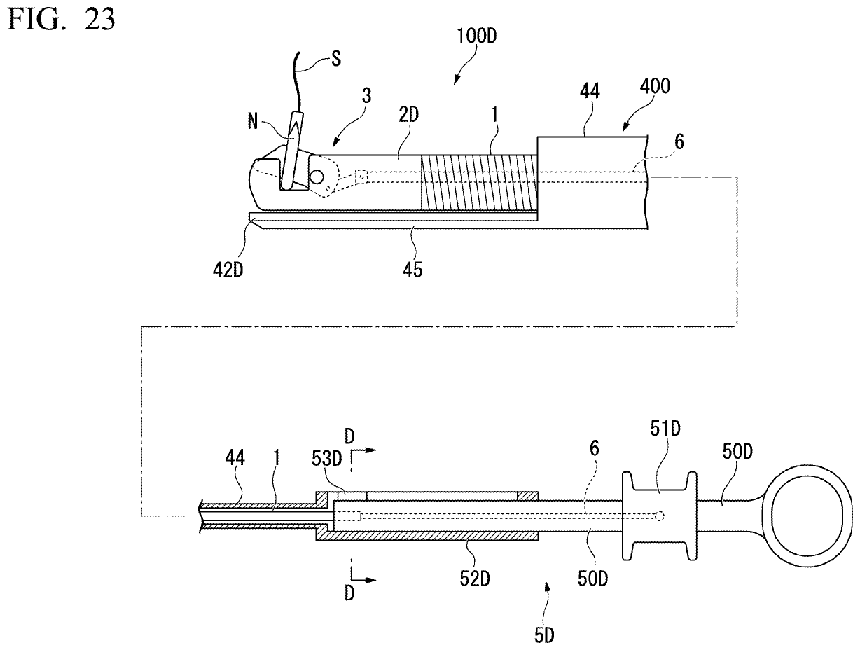

FIG. 23 is a view illustrating the needle holder 100D and the outer sheath 400.

The needle holder (first holder, first holder) 100D includes the sheath 1, a hard portion 2D, the grasper (first holder) 3, an operation portion 5D, and the first operation wire 6 for operating the grasper 3.

The hard portion 2D is similar to the hard portion 2 of the first embodiment, except that the penetration hole 21 through which the elongated member 4 can be inserted is not formed therein.

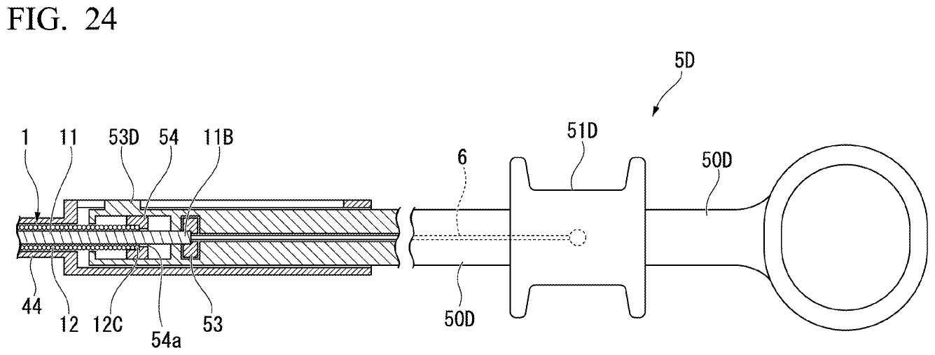

FIG. 24 is a view illustrating a connection part between the sheath 1 and the operation portion 5D in a cross-sectional view.

The operation portion (handle) 5D has an operation portion train body 50D, a first slider 51D, a sheath handle 52D, the engage'member (disk) 53, and the sliding member (key) 54.

Similar to the first embodiment, the proximal end 12C of the second coil sheath 12 is fixed to the sliding member 54 inside the operation portion main body 50D. The proximal end 11B of the first coil sheath 11 extending from the second coil sheath 12 is fixed to the engage member 53.

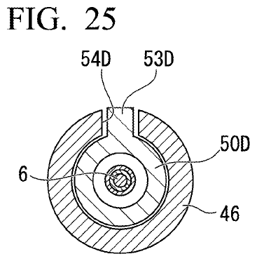

FIG. 25 is a cross-sectional view along line D-D in FIG. 23.

The operation portion main body 50D is formed to have substantially a cylindrical shape, and the first operation wire 6 is inserted through the inside thereof. A projection portion 53D extending in the axial direction of the operation portion main body 50D is formed in an outer circumference of the operation portion main body 50D.

The first slider 51D is coupled so as to be advanceable and retractable with respect to the operation portion main body 50D. The first slider 51D is advanceable and retractable in the axial direction of the operation portion main body 50D. The first operation wire 6 extending from the first coil sheath 11 is connected to the first slider 51D through the inside of the operation portion main body 50D.

The sheath handle 52D is slidably attached to the outer circumference of the operation portion main body 50D and is capable of advancing and retracting in the axial direction of the operation portion main body 50D. The sheath handle 52D has a slit 54D extending in the axial direction of the sheath handle 52D and engaging with the projection portion 53D. The sheath handle 52D is attached to the proximal end of the cylinder member 44 of the outer sheath 400.

[Method for Using Traction System 300D]

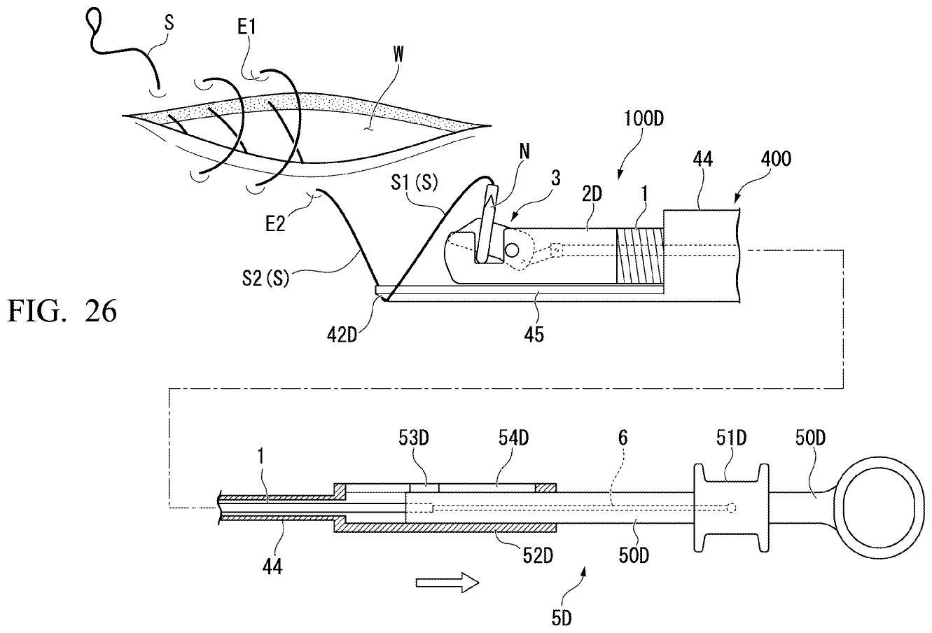

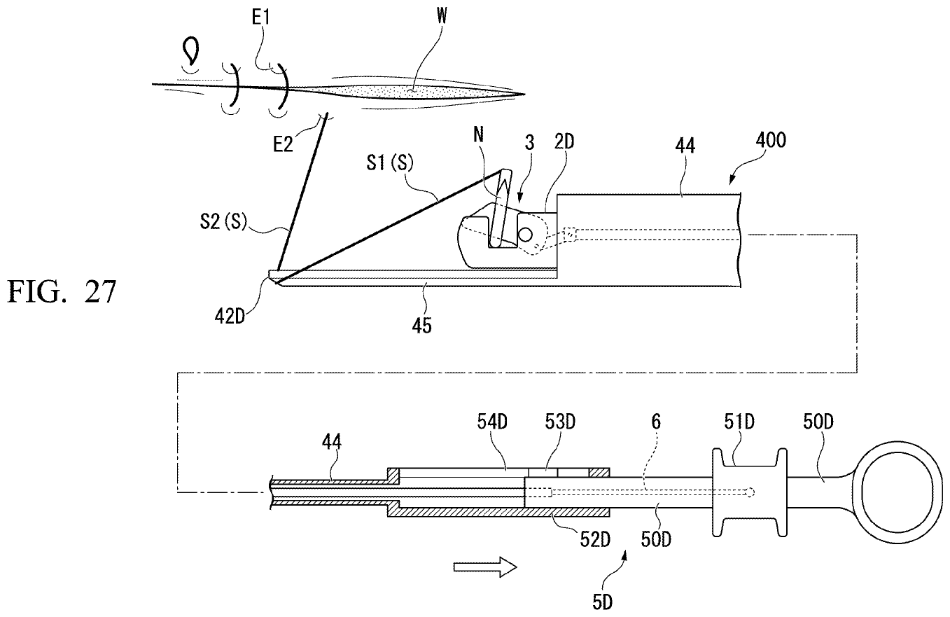

Next, with reference to FIGS. 26 and 27, a procedure using the traction system 300D of the present embodiment (method for using the traction system 300D) will be described. Similar to the first embodiment, a procedure of suturing the wound W formed in the lumen wall T inside a hollow organ such as the digestive tract using the traction system 300D will be described. FIGS. 26 and 27 are views illustrating the method for using the traction system 300D.

The operator causes the outer sheath 400 through which the needle holder 100D is inserted to be inserted through the channel 206 before the flexible endoscope 200 is inserted into a hollow organ. The operator causes the grasper 3 of the needle holder 100D to protrude through the distal end opening portion 206a of the channel 206 of the flexible endoscope 200. The operator grasps the suture needle N with the grasper 3.

The operator inserts the flexible endoscope 200 into a hollow organ through a natural opening of a patient. It is preferable that the flexible endoscope 200 is inserted into a hollow organ in a state in which the suture needle N is stored inside the outer sheath 400. The suture needle N is introduced into a hollow organ in a state of being grasped by the grasper 3 which has protruded through the distal end opening portion 206a.

[First Step and Second Step]

Similar to the first embodiment, the operator performs the first step and the second step.

[Third Step]

FIG. 26 is a view for explaining the third step.

In the third step, while holding the first part S1 of the suture thread S extending from the second edge portion E2 or the suture needle N with the grasper (first holder) 3, the operator engages (holds) the second part 52 of the suture thread S extending between the suture needle N or the first part S1 and the second edge portion E2 in the distal end portion of the outer sheath 400 so, as to be advanceable and retractable. Specifically, the operator engages the suture thread S so as to be advanceable and retractable with the hook portion 42D formed at a distal end of the extension portion 45 of the outer sheath 400.

[Fourth Step]

FIG. 27 is a view for explaining the fourth step.

In the fourth step, the operator pulls the suture thread S by relatively moving the grasper (first holder) 3 of the needle holder (first holder) 100D and the extension portion (second holder) 45 of the outer sheath (second holder) 400. Specifically, the operator advances the outer sheath 400 with respect to the grasper 3 until the distal end (hook portion 42D) of the extension portion 45 of the outer sheath is positioned closer to the distal side than the grasper 3 by moving the sheath handle 52D toward the distal end side with respect to the operation portion main body 50D. The hook portion 42D of the extension portion (second holder) 45 in which the suture thread S has been engaged moves in a direction in which the hook portion 42D becomes more distant from the grasper (first holder) 3 grasping the suture needle N and the second edge portion E2. As a result, a tension is applied to the suture thread S and therefore the first edge portion E1 and the second edge portion E2 are tightened.

According to the traction system 300D according to the present embodiment, for example, a wound can be sufficiently stitched by simply pulling the suture thread S attached to the suture needle N in suturing work inside a confined hollow organ such as a part in the vicinity of the pyloric part of the stomach.

According to the procedure using the traction system 300D (method of use) according to the present embodiment, when the suture thread S attached to the suture needle N is pulled, there is no need to move the grasper 3 grasping the suture needle N. For this reason, it is easy for the operator to restart suturing work of the wound W after traction of the suture thread S.

Hereinabove, the second embodiment of the present disclosure has been described with reference to the drawings, but specific constitutions are not limited to this embodiment, and design change and the like within a range not departing from the gist of the present disclosure are also included therein. In addition, the constituent elements illustrated in the embodiment and the modified examples described above can be constituted to be suitably combined.

### MODIFIED EXAMPLE 3

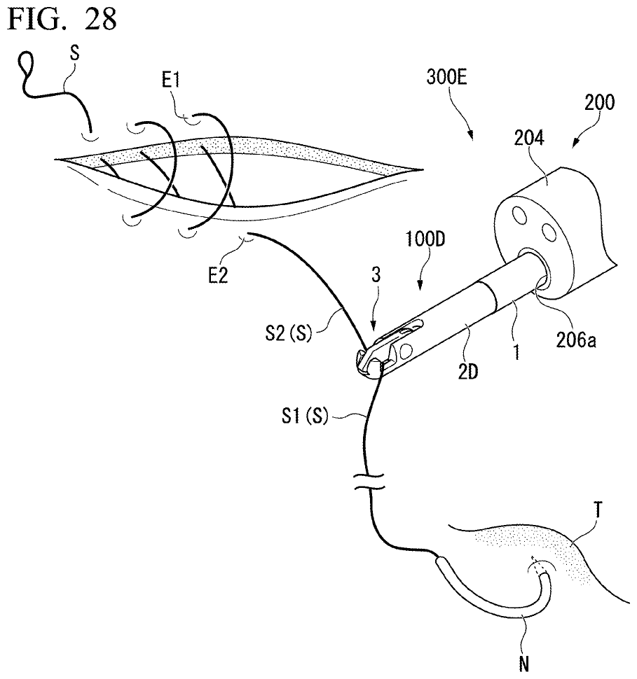

FIG. 28 is a view illustrating a traction system 300E that is a modified example of the traction system 300D. The traction system 300E includes the flexible endoscope (guide device, second holder) 200 and the, needle holder (first holder) 100D. Compared to the traction system 300D, the traction system 300E does not include the outer sheath 400.

The operator performs the first step and the second step similar to those in the foregoing embodiment. In the third step, the operator fixes the suture needle N by puncturing a part of the lumen wall T with the suture needle N. Next, the operator grasps (holds) the first part S1 of the suture thread S extending between the suture needle N and the second edge portion E2 with the grasper (first holder) 3 of the needle holder 100D so as to be advanceable and retractable.

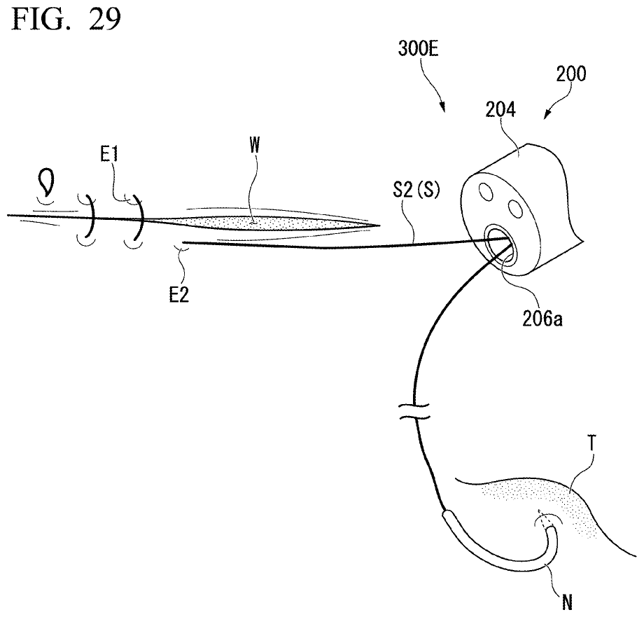

FIGS. 29 and 30 are explanatory views of the fourth step performed using the traction system 300E. In the fourth step, the operator pulls the suture thread S by relatively moving the needle holder (first holder) 100D and the flexible endoscope (second holder) 200. Specifically, the operator moves'the grasper 3 of the needle holder 100D inside the channel 206 of the flexible endoscope 200 by moving the grasper 3 of the needle holder 100D toward the proximal side of the flexible endoscope (second holder) 200. The grasper (first holder) 3 of the needle holder (first holder) 100D grasping the suture thread S moves in a direction in which the grasper 3 becomes more distant with respect to the distal end opening portion 206a of the flexible endoscope (second holder) 200. As a result, a tension is applied to the suture thread S hooked to the distal end opening portion 206a, and the first edge portion E1 and the second edge portion E2 are tightened. Inside the channel 206 of the flexible endoscope 200, the suture thread S is folded back with the first part S1 grasped by the grasper (first holder) 3 as a base point, and therefore it is possible to secure a more sufficient traction amount than pulling the suture needle N fixed to the end portion of the suture thread S.

According to the traction system 300E that is a modified example of the traction system 300D, even if the outer sheath 400 is not included therein, for example, a wound can be sufficiently stitched by simply pulling the suture thread S in suturing work inside a confined hollow organ such as a part in the vicinity of the pyloric part of the stomach.

### MODIFIED EXAMPLE 4

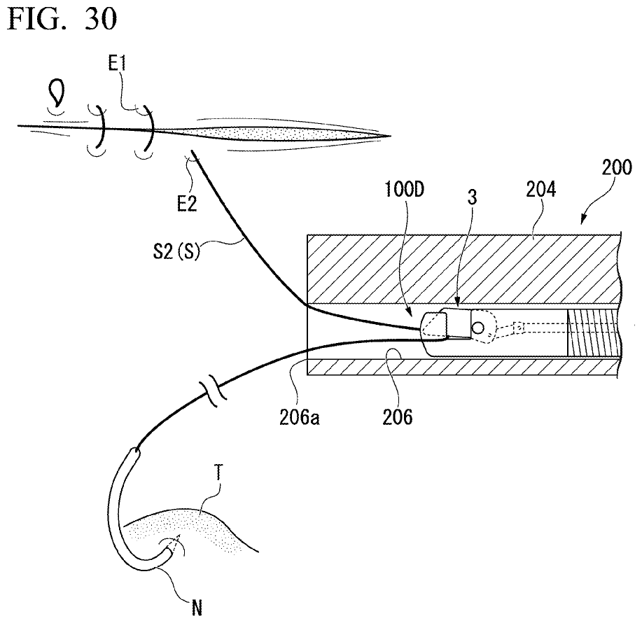

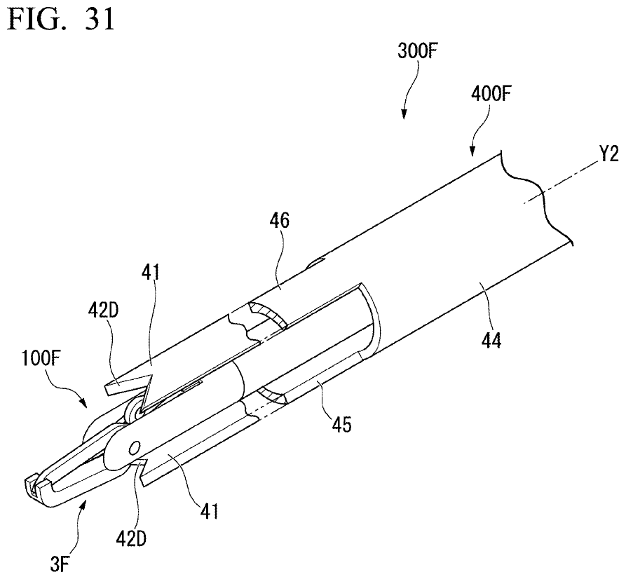

FIG. 31 is a view illustrating a traction system 300F that is a modified example of the traction system 300D. The traction system 300F includes the flexible endoscope 200, a needle holder 100F, and an outer sheath 400F. The needle holder 100F is used so as to be inserted into the flexible endoscope 200 in a state of being inserted through the outer sheath 400F.

The outer sheath (second holder) 400F is the cylinder member (guide member) 44. The outer sheath 400F has the extension portion (second holder) 45 and a second extension portion (second holder) 46 at the distal end of the cylinder member 44. The second extension portion 46 extends in the same direction as the extension portion 45 and is provided at the distal end of the cylinder member 44. The extension portion 45 and the second extension portion 46 are disposed on both sides with the longitudinal axis Y3 of the outer sheath 400 interposed therebetween. The extension portion 45 and the second extension portion 46 are formed to have a width smaller than the outer diameter of the cylinder member 44 and extend from the edge of the distal end opening of the cylinder member 44 along the longitudinal axis Y3 of the outer sheath 400. The shapes of the transverse sections of the extension portion 45 and the second extension portion 46 may have an arc shape or may have a circular shape or a plate shape. The distal end portions 41 of the extension portion 45 and the second extension portion 46 each have the hook portion (engaging surface) 42D in which the suture thread S is capable of being engaged so as to be advanceable and retractable. The hook portion 42D is configured to release an engagement with the suture thread. The hook portion 42D has a recessed shape, and it may have a U-shape or a V-shape, for example.

The needle holder (first holder) 100F is obtained by replacing the grasper 3 in the needle holder 100 of the second embodiment with a double-opening grasper 3F. The grasper 3F is suitably selected from known double-opening grasp members.

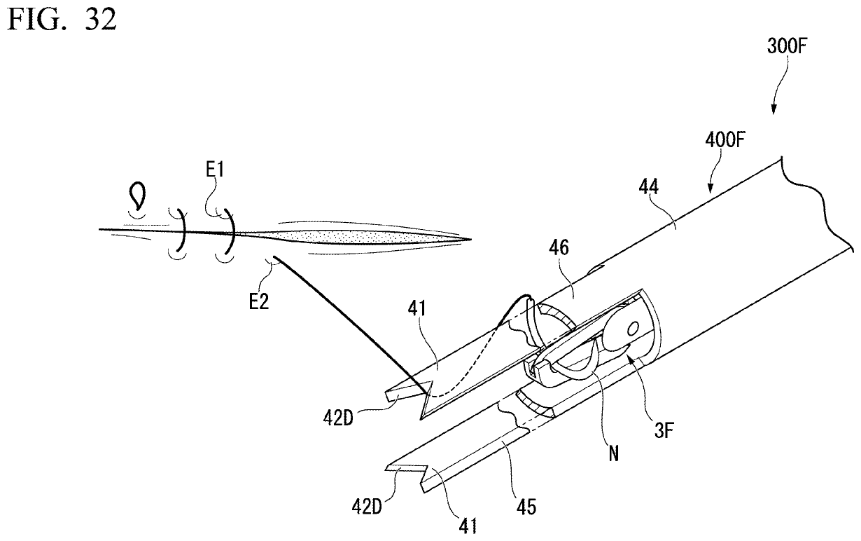

FIG. 32 is a view for explaining the fourth step performed using the traction system 300F.

The operator performs the first step to the third step similar to those in the foregoing embodiment. In the fourth step, the operator pulls the suture thread S by relatively moving the grasper (first holder) 3F of the needle holder (first holder) 100D, and the extension portion (second holder) 45 and the second extension portion (second holder) 46 of the outer sheath (second holder) 400F. Specifically, the operator advances the outer sheath 400F with respect to the grasper 3 by moving the sheath handle 52D toward the distal end side with respect to the operation portion main body 50D. In accordance with this operation, the hook portions 42D of the extension portion (second holder) 45 and the second extension portion (second holder) 46 in which the suture thread S has been engaged moves in a direction in which they become more distant from the grasper (first holder) 3 grasping the suture needle N and the second edge portion E2. As a result, a tension is applied to the suture thread S, and therefore the first edge portion E1 and the second edge portion E2 are tightened.

According to the traction system 300F that is a modified example of the traction system 300D, the outer sheath 400F has two members for holding the second part S2 of the suture thread S, and therefore it is easy to perform treatment in the fourth step.

### MODIFIED EXAMPLE 5

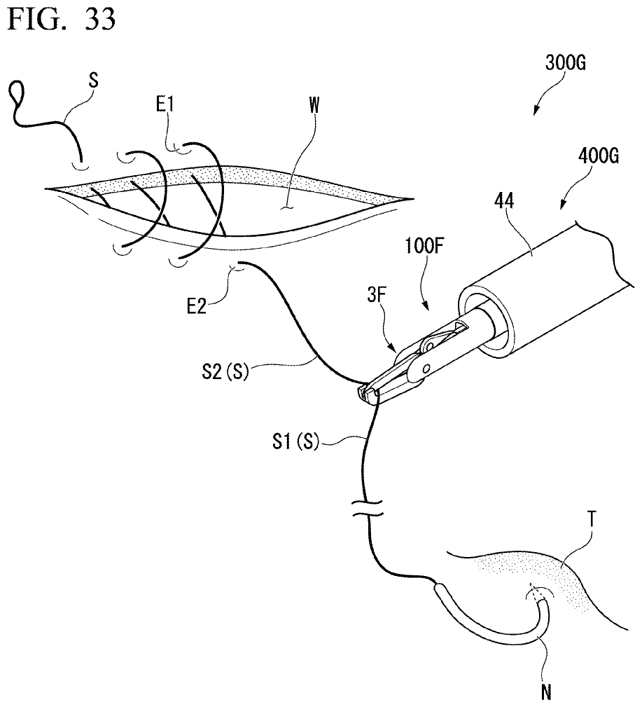

FIG. 33 is a view illustrating a traction system 300G that is a modified example of the traction system 300D. The traction system 300G includes the flexible endoscope 200, the needle holder 100F, and an outer sheath 400G. The outer sheath (second holder) 400G is the cylinder member 44 but does not have the extension portion 45 at the distal end of the cylinder member 44.

The operator performs the first step and the second step similar to those in the foregoing embodiment. In the third step, the operator fixes the suture needle N by puncturing a part of the lumen wall T with the suture needle N. Next, the operator grasps (holds) the first part S1, which extends between the suture needle N and the second edge portion E2, of the suture thread S with the grasper 3 of the needle holder (first holder) 100F so as to be advanceable and retractable.

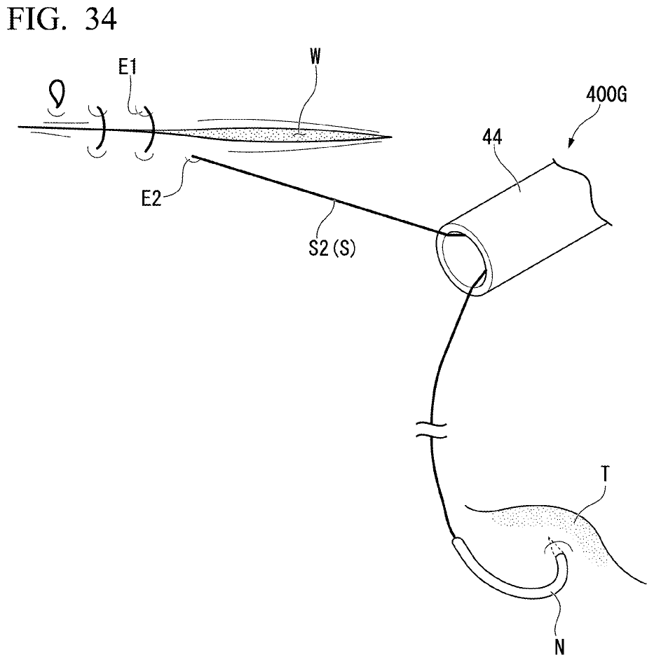

FIG. 34 is a view for explaining the fourth step performed using the traction system 300G.

In the fourth step, the operator pulls the suture thread S by relatively moving the needle holder (first holder) 100D and the outer sheath (second holder) 400G. Specifically the operator moves the grasper 3 of the needle holder 100D inside the cylinder member 44 of the outer sheath 400G by moving the grasper 3F of the needle holder 100F rearward with respect to the outer sheath 400G. The grasper 3 of the needle holder (first holder) 100D grasping the suture thread S moves in a direction in which the grasper 3 becomes more distant with respect to the distal end opening of the outer sheath (second holder) 400G. As a result, a tension is applied to the suture thread S hooked to the outer sheath 400G, and therefore the first edge portion E1 and the second edge portion E2 are tightened. Inside the outer sheath (second holder) 400G, the suture thread S is folded back with the first part S1 grasped by the grasped (first holder) 3F as a base point, and therefore it is possible to secure a more sufficient traction amount than pulling the suture needle N fixed to the end portion of the suture thread S.

According to the traction system 300G that is a modified example of the traction system 300D, even if the outer sheath 400G does not have the extension portion 45, for example, a wound can be sufficiently stitched by simply pulling the suture thread S in suturing work inside a confined hollow organ such as a part in the vicinity of the pyloric part of the stomach.

### MODIFIED EXAMPLE 6

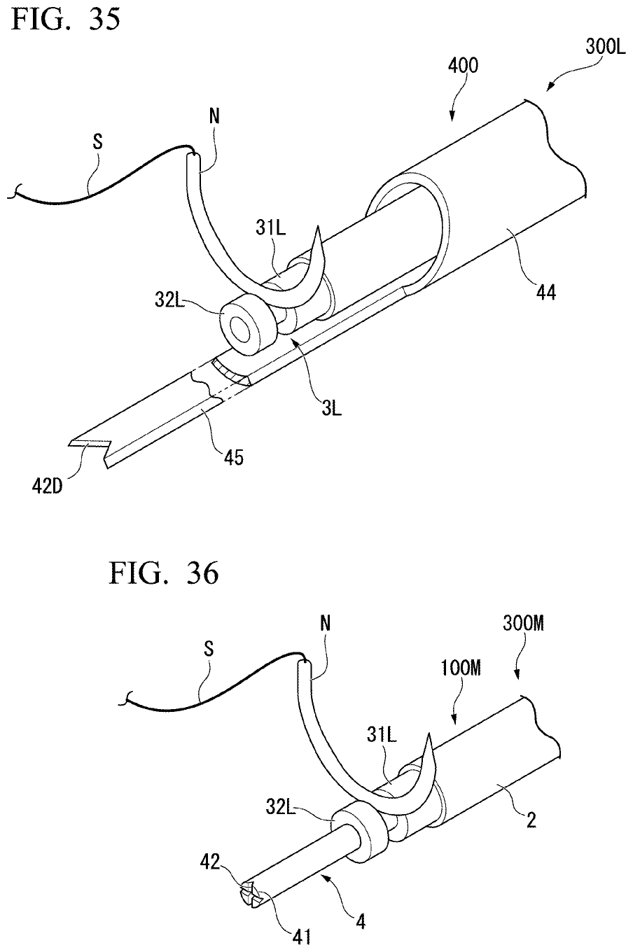

FIG. 35 is a view illustrating a traction system 300L that is a modified example of the traction system 300D. The traction system 300L includes the flexible endoscope 200, a needle holder 100L, and the outer sheath 400.

The needle holder 100L includes the sheath 1, the hard portion 2D, a grasper (first holder) 3L, the operation portion 5D, and the first operation wire 6 for operating the grasper 3L.

The grasper (first holder) 3L has a first grasp member 31L and a second grasp member 32L. The second grasp member 32L is provided closer to the distal side than the first grasp member 31L and is capable of advancing and retracting with respect to the first grasp member 31L. The grasper (first holder) 3L can grasp the suture needle N or the suture thread S by sandwiching it between the first grasp member 31L and the second grasp member 32L.

### MODIFIED EXAMPLE 7

FIG. 36 is a view illustrating a traction system 300M that is a modified example of the traction system 300D. The traction system 300M includes the flexible endoscope 200 and a needle holder 100M.

The needle holder 100M includes the sheath 1, the hard portion 2, the grasper (first holder) 3L, the elongated member (second holder) 4, the operation portion 5D, the first operation wire 6 for operating the grasper 3L, and the second operation wire 7 for operating the elongated member 4. The elongated member 4 penetrates the grasper 3L along the longitudinal axis Y1, and the distal end portion 41 of the elongated member 4 can come in and out through the distal end of the grasper 3L.

In the fourth step, the operator pulls the suture thread S by relatively moving the needle holder (first holder) 100M holding the suture thread S and the groove 42 of the elongated member (second holder) 4 in which the suture thread S has been engaged.

### Third Embodiment

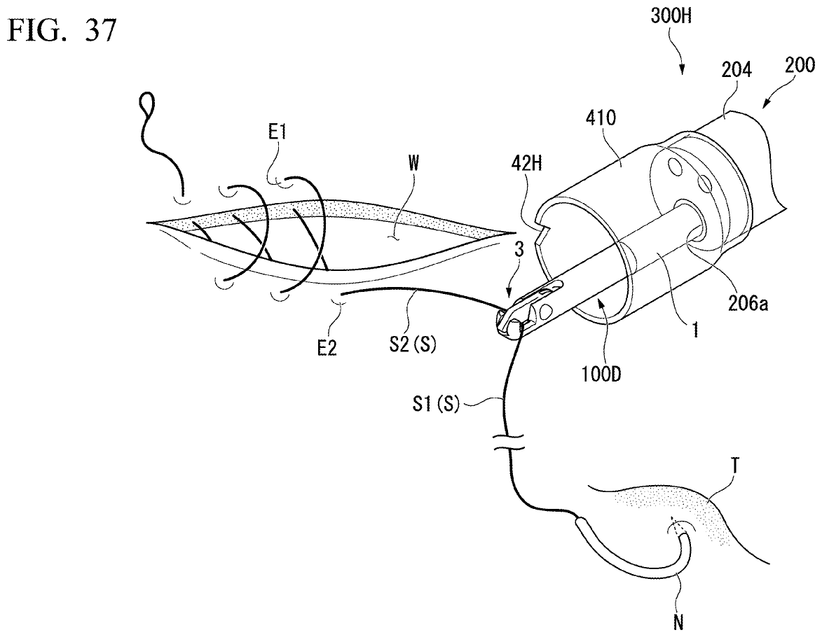

A traction system 300H according to a third embodiment of the present disclosure will be described with reference to FIGS. 37 to 40. In the following description, the same reference signs are applied to constituents common to those which have already been described, and duplicate description will be omitted.

[Traction System 300H]

FIG. 37 is a perspective view illustrating a distal end portion of the traction system 300H.

The traction system 300H includes the flexible endoscope (guide device) 200, the needle holder (first holder) 100D, and a cap (second holder) 410.

The cap (second holder, tube) 410 is a cap which can be mounted at a distal end of the flexible endoscope 200. The cap 410 is formed to have substantially a cylindrical shape in which the proximal end side is slightly reduced in diameter. A proximal end side of the cap 410 can be mounted at the distal end of the flexible endoscope 200. A hook portion (engaging surface) 42H is formed on the distal end side of the cap 410. Regarding the cap 410, a cap suitably selected from known endoscopic caps can be used.

[Method for Using Traction System 300H]

Next, a procedure using the traction system 300H of the present embodiment (method for using the traction system 300H) will be described. Similar to the first embodiment, a procedure of suturing the wound W formed in the lumen wall T inside a hollow organ such as the digestive tract using the traction system 300H will be described.

The operator causes the grasper 3 of the needle holder 100D to protrude through the distal end opening portion 206a of the channel 206 of the flexible endoscope 200 before the flexible endoscope 200 is inserted into a hollow organ. The operator grasps the suture needle N with the grasper 3.

The operator inserts the flexible endoscope 200 into a hollow organ through a natural opening of a patient. It is preferable that the flexible endoscope 200 be inserted into a hollow organ in a state in which the suture needle N is stored inside the cap 410. The suture needle N is introduced into a hollow organ in a state of being grasped by the grasper 3 which has protruded through the distal end opening portion 206a.

[First Step and Second Step]

Similar to the first embodiment, the operator performs the first step and the second step.

[Third Step]

As illustrated in FIG. 37, in the third step, the operator fixes the suture needle N by puncturing a part of the lumen wall T with the suture needle N. Next, the operator grasps (holds) the first part S1, which extends between the suture needle N and the second edge portion E2, of the suture thread S with the grasper 3 of the needle holder (first holder) 100D so as to be advanceable and retractable.

[Fourth Step]

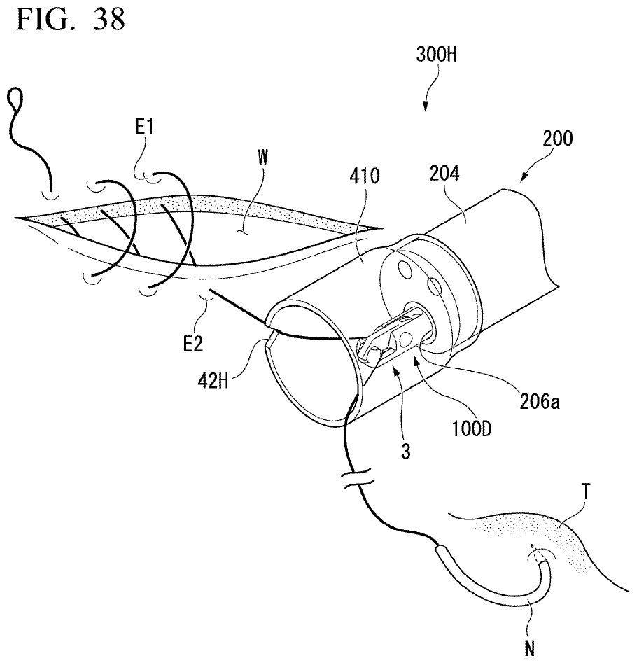

FIGS. 38 to 40 are explanatory views of the fourth step performed using the traction system 300H. In the fourth step, the operator pulls the suture thread S by relatively moving the needle holder (first holder) 100D and the cap (second holder) 410. As illustrated in FIG. 38, the operator moves the grasper 3 of the needle holder 100D inside the cap 410 by moving the grasper 3 of the needle holder 100D rearward with respect to the flexible endoscope 200. At this time, it is preferable that the second part S2 of the suture thread S is hooked to the hook portion 42H of the cap 410 so as to be advanceable and retractable.

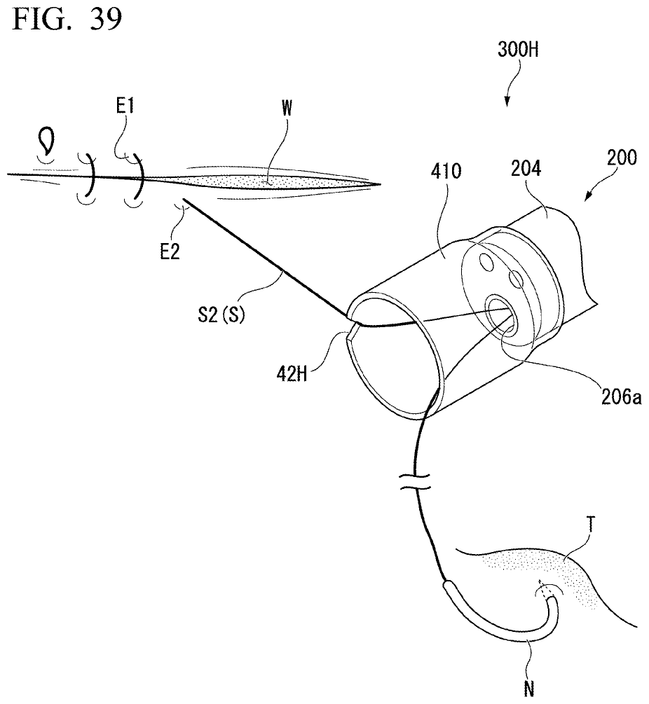

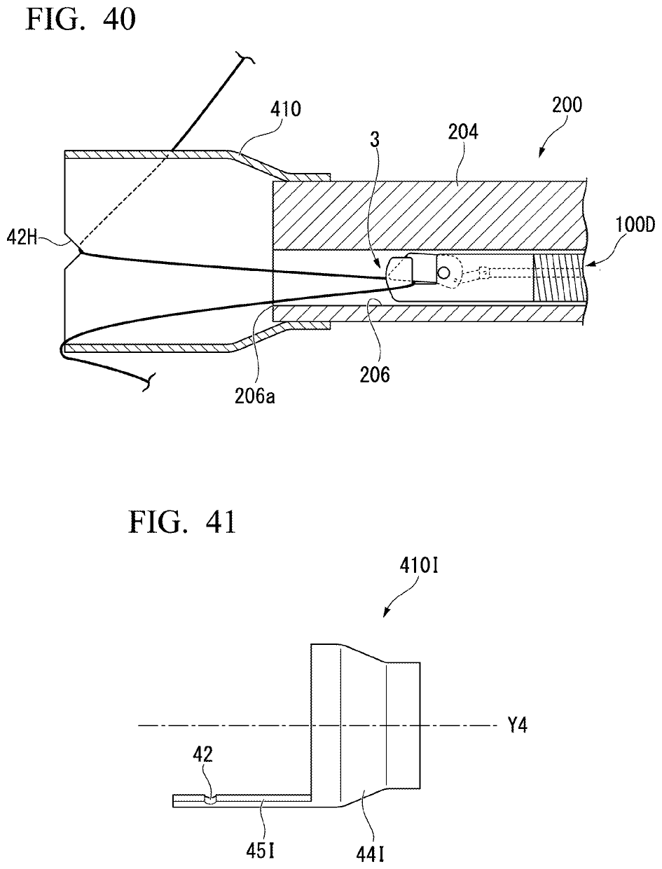

As illustrated in FIGS. 39 and 40, the operator moves the grasper 3 of the needle holder 100D inside the channel 206 of the flexible endoscope 200 by further moving the grasper 3 of the needle holder 100D rearward. The grasper 3 of the needle holder (first holder) 100D grasping the suture thread S moves in a direction in which it becomes more distant with respect to the distal end opening of the cap (second holder) 410. As a result, a tension is applied to the suture thread S hooked to the cap 410, and therefore the first edge portion E1 and the second edge portion E2 are tightened. Since the suture thread S is hooked to the hook portion 42H, positional misalignment of an engagement position between the suture thread S and the cap 410 is unlikely to occur. Inside the channel 206 of the flexible endoscope 200, the suture thread S is folded back with the first part S1 grasped by the grasper (first holder) 3 as a base point, and therefore it is possible to secure a more sufficient traction amount than pulling the suture needle N fixed to the end portion of the suture thread S.

According to the procedure using the traction system 300H and the traction system 300H (method of use) according to the present embodiment, by combining the flexible endoscope 200, the needle holder 100D, and the cap 410, it is possible to easily realize a procedure of sufficiently stitching a wound by simply pulling the suture thread S attached to the suture needle N, for example, in suturing work inside a confined hollow organ such as a part in the vicinity of the pyloric part of the stomach.

Hereinabove, the third embodiment of the present disclosure has been described with reference to the drawings, but specific constitutions are not limited to this embodiment, and design change and the like within a range not departing from the gist of the present disclosure are also included therein. In addition, the constituent elements illustrated in the embodiment and the modified examples described above can be constituted to be suitably combined.

### MODIFIED EXAMPLE 8

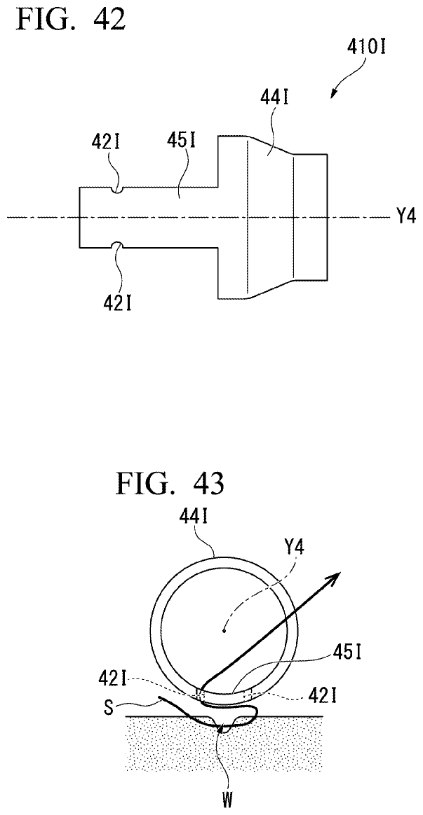

FIG. 41 is a side view of a cap 410I that is a modified example of the cap 410. FIG. 42 is a side view of the cap 410I illustrated in FIG. 41 rotated 90 degrees around the axis. The cap (second holder) 410I has a cylinder member 44I and a plate member 45I provided at the distal end of the cylinder member 44I. The cylinder member 44I is formed to have substantially a cylindrical shape in which the proximal end side is slightly reduced in diameter. The proximal end side of the cylinder member 44I can be mounted at the distal end of the flexible endoscope 200. The plate member 45I extends along a longitudinal axis Y4 of the cap 410I. The plate member 45I has a recessed hook portion 42I in which the suture thread S can be engaged at the distal end. As illustrated in FIG. 42, the hook portion 42I is formed in both end portions in a short-length direction of the plate member 45I.

FIG. 43 is a view for explaining the third step performed using the cap 410I.

In the third step, the operator rotates the distal end of the plate member 45I of the cap 410I about the longitudinal axis Y4 of the cap 410I by vertically and laterally bending the bent portion 204 of the flexible endoscope 200. The suture thread S is wound around the plate member 45I, and a part of the suture thread S is engaged with the hook portion 42I. The operator engages (holds) the second part S2, which extends between the suture needle N and the second edge portion E2, of the suture thread S with the hook portion 42I of the plate member 45I of the cap (second holder) 410I so as to be advanceable and retractable.

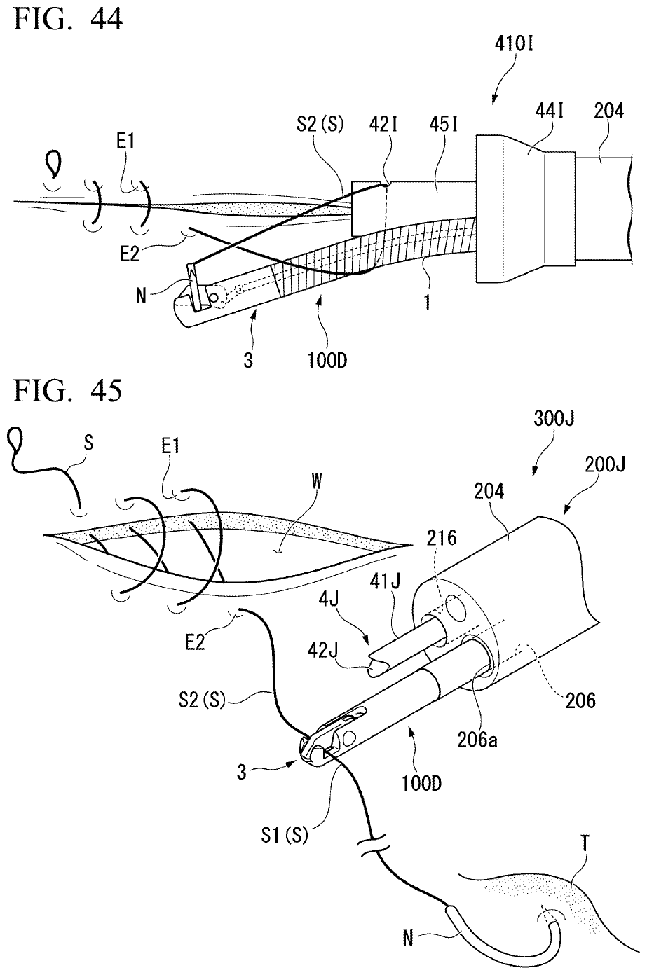

FIG. 44 is a view for explaining the fourth step performed using the cap 410I.

In the fourth step, the operator pulls the suture thread S by relatively moving the needle holder (first holder) 100D and the cap (second holder) 410I. Specifically, the operator advances the needle holder 100D with respect to the flexible endoscope in which the cap 410I is mounted. The hook portion 42I of the cap (second holder) 410I in which the suture thread S has been engaged moves in a direction in which it becomes more distant from the grasper (first holder) 3 grasping the suture needle N and the second edge portion E2. As a result, a tension is applied to the suture thread S, and therefore the first edge portion E1 and the second edge portion E2 are tightened.

### Fourth Embodiment

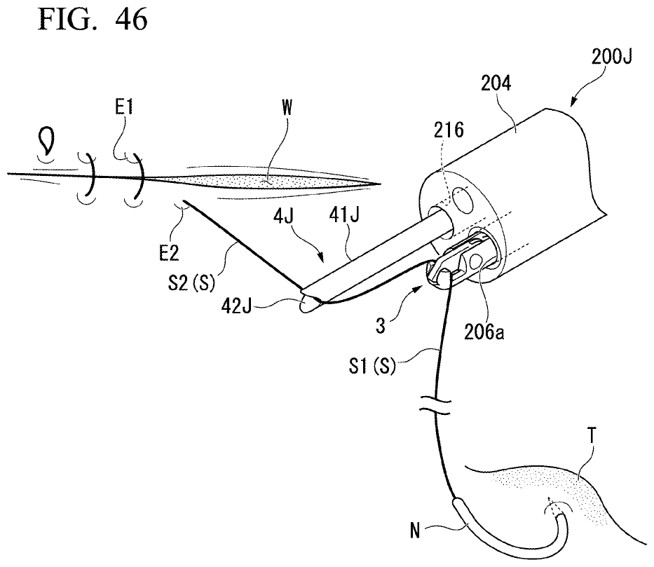

A traction system 300J according to a fourth embodiment of the present disclosure will be described with reference to FIGS. 45 and 46. In the following description, the same reference signs are applied to constituents common to those which have already been described, and duplicate description will be omitted.

[Traction System 300J]

FIG. 45 is a perspective view illustrating a distal end portion of the traction system 300J.

The traction system 300J includes a flexible endoscope (guide device) 200J, the needle'holder (first holder) 100D, and an elongated member (second holder) 4J.

The flexible endoscope (guide device) 200J is a so-called two-channel endoscope and additionally has a second channel 216 in addition to the channel 206.

The elongated member (second holder) 4J is formed to have a columnar shape and is inserted through the second channel 216 of the flexible endoscope 200J. A recessed hook portion (engaging surface) 42J is formed at the distal end of the elongated member 4J.

[Method for Using Traction System 300J]

Next, a procedure using the traction system 300J of the present embodiment (method for using the traction system 300J) will be described. Similar to the first embodiment, a procedure of suturing the wound W formed in the lumen wall T inside a hollow organ such as the digestive tract using the; traction system 300J will be described.

Similar to the third embodiment, the operator introduces the suture needle N into a hollow organ in a state of being grasped by the grasper 3 of the needle holder 100D which has protruded through the distal end opening portion 206a.

[First Step and Second Step]

Similar to the third embodiment, the operator performs the first step and the second step.

[Third Step]

As illustrated in FIG. 45, the operator causes a distal end portion 4IJ of the elongated member 4J to protrude through the distal end opening of the second channel 216. In the third step, the operator fixes the suture needle N by puncturing a part of the lumen wall T with the suture needle N. Next, in the third step, while holding the first part S1 of the suture thread S extending from the second edge portion E2 with the grasper (first holder) 3 of the needle holder (first holder) 100D, the operator engages (holds) the second part S2, which extends between the first part S1 and the second edge portion E2 of the suture thread S in the distal end portion of the elongated member (second holder) 4J so as to be advanceable and retractable. Specifically, the operator engages the suture thread S with the hook portion 42J of the elongated member (second holder) 4J so as to be advanceable and retractable.

[Fourth Step]

FIG. 46 is a view for explaining the fourth step performed using the traction system 300J.

In the fourth step, the operator pulls the suture thread S by relatively moving the needle holder (first holder) 100D and the elongated member (second holder) 4J. Specifically, the operator advances the elongated member 4J with respect to the grasper 3 until the distal end portion 41J (hook portion 42J) of the elongated member 4J is positioned closer to the distal side than the grasper 3. In accordance with this operation, the hook portion 42J of the, elongated member (second holder) 4J in which the suture thread S has been engaged moves in a direction in which it becomes more distant from the grasper (first holder) 3 grasping the suture thread S and the second edge portion E2. As a result, a tension is applied to the suture thread S, and therefore the first edge portion E1 and the second edge portion E2 are tightened.

According to the procedure using the traction system 300J and the traction system 300J (method of use) according to the present embodiment, by combining the flexible endoscope 200J (two-channel endoscope), the needle holder 100D, and the elongated member 4J, it is possible to easily realize a procedure of sufficiently stitching a wound by simply pulling the suture thread S attached to the suture needle N, for example, in suturing work inside a confined hollow organ such as a part in the vicinity of the pyloric part of the stomach.

### INDUSTRIAL APPLICABILITY

The present disclosure can be applied to medical instruments for suturing a wound or the like inside a hollow organ.

Although the present disclosure has been described in connection with preferred embodiments thereof, it will be appreciated by those skilled in the art that additions, deletions, modifications, and substitutions, not specifically described may be made without department from the spirit and scope of the invention as defined in the appended claims.

What is claimed is:

1. A traction tool for performing traction of a suture thread under a flexible endoscope, the traction tool comprising:
a first holder configured to hold the suture thread or a′suture needle attached to the suture thread;
a second holder having an engaging surface configured to engage the suture thread such that the suture thread is capable of advancing and retracting and configured to release an engagement with the suture thread; and
a guide member configured to hold the first holder and the second holder such that the first holder and the second holder are relatively movable.

2. The traction tool according to claim 1 further comprising:
a sheath; and
a hard portion provided at a distal end of the sheath,
wherein the first holder is a needle holder having a first grasp member disposed in the hard portion and a second grasp member provided in the hard portion so as to be turnable, and the first holder is configured to grasp a needle, which is attached to the suture thread, between the first grasp member and the second grasp member, and
wherein the second holder is an elongated member which is inserted through the hard portion.

3. The traction tool according to claim 1,
wherein the engaging surface is a groove formed on an end surface of the distal end portion.

4. The traction tool according to claim 2,
wherein the engaging surface is a protrusion extending in a direction intersecting a longitudinal axis of the elongated member.

5. The traction tool according to claim 1,
wherein the first holder is a needle holder. and
wherein the second holder is an outer sheath into which the needle holder is inserted.

6. The traction tool according to claim 5,
wherein the outer sheath has an extension portion formed at a distal end of the outer sheath, and
wherein the extension portion has a recessed engaging surface.

7. A traction system for performing traction of a suture thread under a flexible endoscope, the traction system comprising:
a sheath;
a hard portion provided at a distal end of the sheath;
a first holder having a first grasp member disposed in the hard portion and a second grasp member provided in the hard portion so as to be turnable, and the first holder being configured to grasp a needle, which is attached to the suture thread, between the first grasp member and the second grasp member; and
a second holder inserted into the hard portion, and configured to hold the suture thread such that the suture thread is capable of advancing and retracting.

8. The traction tool according to claim 7,
wherein a second holder having an engaging surface configured to engage the suture thread such that the suture thread is capable of advancing and retracting and configured to release an engagement with the suture thread.

9. The traction tool according to claim 8,
wherein the engaging surface is a groove formed on an end surface of the distal end portion.

10. The traction tool according to claim 8,
wherein the engaging surface is a protrusion extending in a direction intersecting a longitudinal axis of the elongated member.

11. The traction tool according, to claim 7,
wherein the first holder is a needle holder, and
wherein the second holder is an outer sheath into which the needle holder is inserted.

12. The traction tool according to claim 10,
wherein the outer sheath has an extension portion formed at a distal end of the outer sheath, and
wherein the extension portion has a recessed engaging surface.

13. A traction method for a suture thread using the tool according to claim 1, the traction method comprising:
pulling the suture thread by relatively moving the first holder and the second holder, the first holder holding the suture thread or a suture needle fixed to the suture thread, and the second holder holding the suture thread such that the suture thread is capable of advancing and retracting.

14. A traction method for a suture thread using the tool according to claim 7, the traction method comprising:
pulling the suture thread by relatively moving the first holder and the second holder, the first holder holding the suture thread or a suture needle fixed to the suture thread, and the second holder holding the suture thread such that the suture thread is capable of advancing and retracting.

15. A suturing method for closing a wound formed in a lumen wall of a hollow organ using a tool including a first holder configured to, hold suture thread or a suture needle attached to the suture thread, and a second holder configured to hold the suture thread such that the suture thread is capable of advancing and retracting, the suturing method comprising:
a first step of threading a first edge portion of the wound with the suture thread;
a second step of threading the suture thread to a second edge portion of the wound, the suture thread extending from the first edge portion, and the second edge portion being existed at a position facing the first edge portion with the wound interposed therebetween;
a third step of fixing the suture needle or a first part of the suture thread extending from the second edge portion to the lumen wall and holding a second part, which extends between the sutures needle or the first portion and the second edge portion, of the suture thread by the second holder such that the suture thread is capable of advancing and retracting; and
a fourth step of pulling the suture thread by relatively moving the first holder and the second holder.

16. The suturing method according to claim 15,
wherein in the third step, the suture needle or the first part of the suture thread extending from the second edge portion is fixed by holding the suture needle or the first part of the suture thread with the first holder.

17. The suturing method according to claim 15,
wherein in the third step, the suture needle or the first part of the suture thread extending from the second edge portion is fixed by threading the lumen wall with the suture needle.

18. The suturing method according to claim 15,
wherein the third step further includes:
a step of forming a loop using the suture thread by rotation operation of the first holder, and
a step of hooking the second holder to the loop by an operation advancing or retracting the second holder, and
wherein in the fourth step, the suture thread is pulled by relatively moving the first holder and the second holder in a state in which the suture thread is held by the first holder and the second holder is hooked to the loop.
